(19) United States
(12) Patent Application Publication
(10) Pub. No.: US 2007/0185184 A1
(43) Pub. Date: Aug. 9, 2007

(54) Carbazole derivatives

(75) Inventors: Gunnar J. Hanson, Chapel Hill, NC (US); Thomas E. Barta, Carrboro, NC (US); Lifeng Geng, Cary, NC (US); James Veal, Apex, NC (US); Kenneth He Huang, Chapel Hill, NC (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Serenex, Inc.

(21) Appl. No.: 11/532,340

(22) Filed: Sep. 15, 2006

Related U.S. Application Data
(60) Provisional application No. 60/718,249, filed on Sep. 16, 2005.

(51) Int. Cl. A61K 31/403; C07D 209/82

(52) U.S. Cl. 514/411; 548/444

(57) ABSTRACT

Disclosed are compounds and pharmaceutically acceptable salts of Formula I: 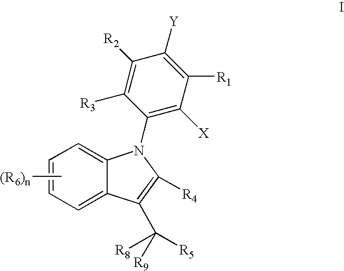
wherein n, R₁, R₂, R₃, X, R₄, R₅, R₆, R₈, R₉, and Y are as defined herein. Compounds of Formula I are useful in the treatment of diseases and/or conditions related to cell proliferation and/or abnormal cell mitosis, such as cancer, inflammation and inflammation-associated disorders, and conditions associated with angiogenesis. Also disclosed are pharmaceutical compositions comprising compounds of the invention and methods of treating the aforementioned conditions using such compounds.

### BACKGROUND OF THE INVENTION

This application claims priority from U.S. Provisional Application Ser. No. 60/718,249 filed Sep. 16, 2005, the disclosure of which is incorporated herein by reference in its entirety.

### FIELD OF THE INVENTION

The invention relates to carbazole derivatives that are useful in the treatment and/or prevention of diseases and/or conditions related to cell proliferation and/or abnormal cell mitosis, such as cancer, inflammation and inflammation-associated disorders, and conditions associated with angiogenesis. More specifically, it relates to compounds that interfere with tubulin polymerization and, as a result, cell mitosis. The invention further relates to methods for treating disease states characterized by abnormal cell mitosis.

### DESCRIPTION OF THE RELATED ART

Cancer treatment can be approached by several modes of therapy, including surgery, radiation, chemotherapy, or a combination of any of these treatments. Among them, chemotherapy is indispensable for inoperable or metastatic forms of cancer.

The microtubule system of eukaryotic cells is an important target for developing anti-cancer agents. More specifically, tubulin polymerization/depolymerization is a popular target for new chemotherapeutic agents. Microtubules show highly dynamic instability and play an important role in cellular mitosis. Chemicals that attack microtubules through their major structural component, tubulin, disrupt or suppress both microtubule structure and normal functions by inhibition or promotion of microtubule assembly. Inhibition or arrest of cellular mitosis is the result.

A variety of clinically used compounds target tubulin polymerization/depolymerization and disrupt cellular microtubule structures, resulting in mitotic arrest.

One example of conventional antimitotic agents includes the vinca alkaloids, which inhibit microtubule polymerization.

Colchicine is another conventional antimitotic agent. Although colchicine has limited medicinal application due to its high toxicity, it has played a fundamental role in elucidation of the properties and fictions of tubulin and microtubules.

Combretastatin A-4 (CA-4) is a potent anti-mitotic agents derived from the stem wood of the South African tree Combretum caffrum. This agent shows strong cytotoxicity against a wide variety of human cancer cells, including multi-drug resistant cancer cells. CA-4, structurally similar to colchicines, possesses a higher affinity for the colchicine binding site on tubulin than colchicine itself. It also has been shown to possess anti-angiogenesis activity. The low water-solubility of CA-4 limits its efficacy in vivo.

Cell mitosis is a multi-step process that includes cell division and replication. Mitosis is characterized by the intracellular movement and segregation of organelles, including mitotic spindles and chromosomes. Organelle movement and segregation are facilitated by the polymerization of the cell protein tubulin. Microtubules are formed from α and β tubulin polymerization and the hydrolysis of GTP. Microtubule formation is important for cell mitosis, cell locomotion, and the movement of highly specialized cell structures such as cilia and flagella.

Numerous diseases are characterized by abnormal cell mitosis. For example, uncontrolled cell mitosis is a hallmark of cancer. In addition, cell mitosis is important for the normal development of the embryo, formation of the corpus luteum, wound healing, inflammatory and immune responses, angiogenesis and angiogenesis related diseases.

Identification of compounds that target the microtubule system (e.g., tubulin polymerization/depolymerization) can lead to new therapeutics useful in treating or preventing cancer or symptoms associated with cancer.

### SUMMARY OF THE INVENTION

In a broad aspect, the invention encompasses the compounds of formula I shown below, pharmaceutical compositions containing those compounds and methods employing such compounds or compositions in the treatment or prevention of diseases and/or conditions related to cell proliferation and/or abnormal cell mitosis, such as cancer, inflammation and inflammation-associated disorders, and conditions associated with angiogenesis.
The invention provides compounds of formula I: 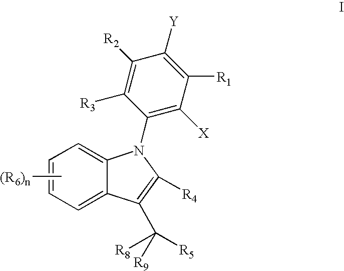
and the pharmaceutically acceptable salts thereof, wherein
X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, mono C₃-C₆ alkenylamino, carboxamide, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
      where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
or R₂ and R₃ together with the atoms to which they are attached form a 5-12 membered mono-, bi-, or tricyclic ring system, where the 5-12 membered ring is partially unsaturated or aromatic and optionally contains one or two of oxygen, S(O)ₘ where m is 0, 1, or 2, nitrogen, or NR₇ where R₇ is hydrogen or C₁-C₆ alkyl, and wherein the 5-12 membered ring is optionally substituted with 1 or 2 R₂₂ groups;
or R₁ and X together with the atoms to which they are attached form a 5-12 membered mono-, bi-, or tricyclic ring system, where the 5-12 membered ring is partially unsaturated or aromatic and optionally contains one or two of oxygen, S(O)ₘ where m is 0, 1, or 2, nitrogen, or NR₇ where R₇ is hydrogen or C₁-C₆ alkyl, and wherein the 5-12 membered ring is optionally substituted with 1 or 2 R₂₂ groups;
Y is H, NR₇R₇′, NR₇(C₃-C₆)alkenyl, NR₇CONHCOR₇′, C(=Z)NR₇R₇′, NR₇CONR₇R₇′, NR₇COR₇′, or —NR₇—(C₁-C₆)alkyl-(C₁-C₆)alkoxy,
   where Z is O, S, or NOR₇, and
   R₇ and R₇′ at each occurrence are independently selected from H and C₁-C₆ alkyl;
R₄ is H or C₁-C₆ alkyl optionally substituted with 1-2 groups selected from oxo, aryl, heteroaryl, or R₂₂;
R₅ is OR₇, NR₇R₇′, NR₇OR₇′, or C₁-C₆ alkyl optionally substituted with 1-2 groups selected from oxo, aryl, heteroaryl, or R₂₂; or
R₄ and R₅ together with the atoms to which they are attached form a 5-12 membered mono-, bi-, or tricyclic ring system, where the 5-12 membered ring is partially unsaturated or aromatic and optionally contains one or two of oxygen, S(O)ₘ where m is 0, 1, or 2, nitrogen, NOR₇ or NR₇ where R₇ is hydrogen or C₁-C₆ alkyl, and wherein the 5-12 membered ring is optionally substituted with 1 or 2 R₂₂ groups;
n is 0, 1, 2, 3, or 4;
R₆ at each occurrence is independently halo, C₁-C₆ alkyl, halo (C₁-C₆)-alkyl, C₁-C₆ alkoxy, nitro, hydroxyl, cyano, alkenyl, alkynyl, thiol, amino, mono- or di-(C₁-C₆) alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl groups and heteroaryl groups is optionally substituted with from 1-4 R₂₂ groups;
or two R₆ groups on adjacent carbons, together with the atoms to which they are attached, form a 5-12 membered mono-, bi-, or tricyclic ring system fused to the ring to which Y is attached, where the 5-12 membered ring is partially unsaturated or aromatic and optionally contains one or two of oxygen, S(O)ₘ where m is 0, 1, or 2, nitrogen, or NR₇ where R₇ is hydrogen or C₁-C₆ alkyl, wherein the 5-12 membered ring is optionally substituted with 1 or 2 R₂₂ groups;
R₈ is H; and
R₉ is OH;
or R₈ and R₉ together are Q, wherein Q is ═O or ═NOR₇, provided that when the 5-12 membered ring formed by R₄ and R₅ is aromatic, one of R₈ and R₉ is absent.

The compounds of the invention have activity as inhibitors of tubulin polymerization. The compounds of the invention are useful as inhibitors of tumor development, as inhibitors of rate of tumor growth, and/or for inducing regression of pre-existing tumors.

The invention also includes intermediates that are useful in making the compounds of the invention.

The invention also provides pharmaceutical compositions comprising a compound or pharmaceutically acceptable salt of Formula I and at least one pharmaceutically acceptable carrier, solvent, adjuvant or diluent.

The invention further provides methods of treating disease related to cell proliferation and/or abnormal cell mitosis, such as cancer, inflammation and inflammation-associated disorders, and conditions associated with angiogenesis, in a patient in need of such treatment, comprising administering to the patient a compound or pharmaceutically acceptable salt of Formula I, or a pharmaceutical composition comprising a compound or salt of Formula I.

The invention also provides the use of a compound or salt according to Formula I for the manufacture of a medicament for use in treating cancer, inflammation and inflammation-associated disorders, and conditions associated with angiogenesis.

The invention also provides methods of preparing the compounds of the invention and the intermediates used in those methods.

The invention further provides a compound or pharmaceutical composition thereof in a kit with instructions for using the compound or composition.

### DETAILED DESCRIPTION OF THE INVENTION

Preferred compounds of Formula I include those wherein X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl or heteroaryl, where each of the foregoing aryl groups and heteroaryl groups is optionally substituted with from 1-4 R₂₂ groups. Also preferred are compounds wherein R₁, R₂, and R₃ are each hydrogen, halo, C₁-C₆ alkyl, C₁-C₆ alkoxy, or hydroxy. Further preferred are compounds wherein X is H, halo, C₁-C₆ alkyl, halo (C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆) alkylamino. More preferred are compounds wherein R₁, R₂, and R₃ are each hydrogen, halo, or C₁-C₆ alkoxy and X is H, halo, C₁-C₆ alkyl, halo(C₁-C₆)-alkyl or C₁-C₆ alkoxy. Still more preferred are compounds wherein R₁, R₂, and R₃ are each hydrogen and X is halo.

Preferred compounds of formula I also include those wherein R₄ is H or C₁-C₆ alkyl and R₅ is OR₇, NR₇R₇′, NR₇OR₇′, or C₁-C₆ alkyl.

Preferred compounds of formula I also include those wherein R₄ and R₅ together with the atoms to which they are attached form a 5-12 membered mono-, bi-, or tricyclic ring system, where the 5-12 membered ring is partially unsaturated, and wherein the 5-12 membered ring is optionally substituted with 1 or 2 R₂₂ groups.

Preferred compounds of formula I are also those wherein n is 0.

Preferred compounds of formula I further include those wherein n is 1 and R₆ is halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl or heteroaryl, where each of the foregoing aryl groups and heteroaryl groups is optionally substituted with from 1-4 R₂₂ groups.

Preferred compounds of formula I further include compounds of formula II: 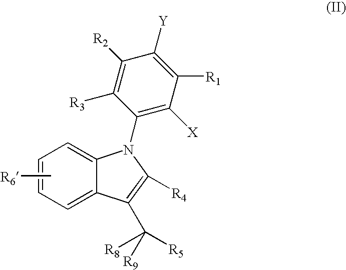
and pharmaceutically acceptable salts thereof, wherein
X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, mono C₃-C₆ alkenylamino, carboxamide, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
      where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
Y is H, NR₇R₇′, NR₇(C₃-C₆)alkenyl, NR₇CONHCOR₇′, —C(=Z)NR₇R₇′, —NR₇CONR₇R₇′, —NR₇COR₇′, or —NR₇—(C₁-C₆)alkyl-(C₁-C₆)alkoxy,
   where Z is O, S, or NOR₇, and
   R₇ and R₇′ at each occurrence are independently selected from H and C₁-C₆ alkyl;
R₄ is H or C₁-C₆ alkyl optionally substituted with 1-2 groups selected from oxo, aryl, heteroaryl, or R₂₂;
R₅ is OR₇, NR₇R₇′, NR₇OR₇′, or C₁-C₆ alkyl optionally substituted with 1-2 groups selected from oxo, aryl, heteroaryl, or R₂₂; or
R₄ and R₅ together with the atoms to which they are attached form a 5-12 membered mono-, bi-, or tricyclic ring system, where the 5-12 membered ring is partially unsaturated and optionally contains one or two of oxygen, S(O)ₘ where m is 0, 1, or 2, nitrogen, NOR₇ or NR₇ where R₇ is hydrogen or C₁-C₆ alkyl, and wherein the 5-12 membered ring is optionally substituted with 1 or 2 R₂₂ groups;
R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
R₈ is H; and
R₉ is OH;
or R₈ and R₉ together are Q, wherein Q is ═O or ═NOR₇.

Preferred compounds of formula II include those wherein X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferably, X, R₁, R₂, and R₃ are independently H, halo, C₁-C₆ alkyl, halo(C₁-C₆)-alkyl or C₁-C₆ alkoxy.

Preferred compounds of formula II also include those wherein R₁, R₂, and R₃ are each hydrogen, halo, C₁-C₆ alkoxy. Further preferred are compounds wherein R₁, R₂, and R₃ are each hydrogen, halo, C₁-C₆ alkoxy and X is H, halo, C₁-C₆ alkyl, halo (C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆) alkylamino. More preferred are compounds wherein R₁, R₂, and R₃ are each hydrogen, halo, C₁-C₆ alkoxy and X is H, halo, nitro, C₁-C₆ alkyl, halo(C₁-C₆)-alkyl or C₁-C₆ alkoxy. Still more preferred are compounds wherein R₁, R₂, and R₃ are each hydrogen and X is H, halo or halo(C₁-C₆)-alkyl.

Preferred compounds of formula II include those wherein Y is CONR₇R₇′, NR₇CONR₇R₇′, or NR₇COR₇′. More preferred are compounds wherein Y is CONH₂, NHCONH₂, or NHCOR₇′. Even more preferred compounds of formula II are those wherein Y is NHCONH₂.

Preferred compounds of formula II also include those wherein R₄ is H or C₁-C₆ alkyl and R₅ is C₁-C₆ alkyl. Preferred compounds are also those wherein R₄ is C₁-C₆ alkyl and R₅ is C₁-C₆ alkyl.

Preferred compounds of formula II further include those wherein R₄ and R₅ together with the atoms to which they are attached form a 5-12 membered mono-, bi-, or tricyclic ring system, where the 5-12 membered ring is partially unsaturated, and wherein the 5-12 membered ring is optionally substituted with 1 or 2 R₂₂ groups. More preferred are compounds wherein R₄ and R₅ together with the atoms to which they are attached form a 6 membered ring, wherein the ring is optionally substituted with 1 or 2 R₂₂ groups.

Preferred compounds of formula II further include those wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, or C₃-C₇ cycloalkyl(C₁-C₆)alkyl. More preferred are compounds wherein R₆′ is H, halo, C₁-C₆ alkyl, halo(C₁-C₆)-alkyl, C₁-C₆ alkoxy, amino, or mono- or di-(C₁-C₆)alkylamino. Even more preferred are compounds wherein R₆′ is H or halo.

Preferred compounds of formula II include those wherein R₈ and R₉ together are Q. Preferably, wherein Q is ═O or ═NOH.

Preferred compounds of formulae I and II further include compounds of formula III: 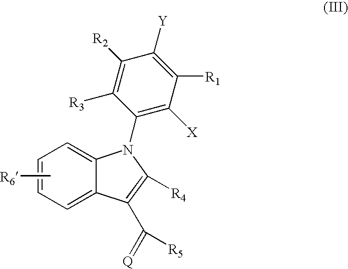
and pharmaceutically acceptable salts thereof, wherein
X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, mono C₃-C₆ alkenylamino, carboxamide, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
      where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
Y is H, NR₇R₇, NR₇(C₃-C₆)alkenyl, NR₇CONHCOR₇′, —C(=Z)NR₇R₇′, —NR₇CONR₇R₇′, —NR₇COR₇′, or —NR₇—(C₁-C₆)alkyl-(C₁-C₆)alkoxy,
   where Z is O, S, or NOR₇, and
   R₇ and R₇′ at each occurrence are independently selected from H and C₁-C₆ alkyl;
R₄ is H or C₁-C₆ alkyl optionally substituted with 1-2 groups selected from oxo, aryl, heteroaryl, or R₂₂;
R₅ is OR₇, NR₇R₇′, NR₇OR₇′, or C₁-C₆ alkyl optionally substituted with 1-2 groups selected from oxo, aryl, heteroaryl, or R₂₂; or
R₄ and R₅ together with the atoms to which they are attached form a 5-12 membered mono-, bi-, or tricyclic ring system, where the 5-12 membered ring is partially unsaturated and optionally contains one or two of oxygen, S(O)ₘ where m is 0, 1, or 2, nitrogen, NOR₇ or NR₇ where R₇ is hydrogen or C₁-C₆ alkyl, and wherein the 5-12 membered ring is optionally substituted with 1 or 2 R₂₂ groups;
R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups; and
Q is O or NOR₇.

Preferred compounds of formula III include those wherein X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferably, X, R₁, R₂, and R₃ are independently H, halo, C₁-C₆ alkyl, halo(C₁-C₆)-alkyl or C₁-C₆ alkoxy.

Preferred compounds of formula III also include those wherein R₁, R₂, and R₃ are each hydrogen. Further preferred are compounds wherein R₁, R₂, and R₃ are each hydrogen, halo, or C₁-C₆ alkoxy and X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferred are compounds wherein R₁, R₂, and R₃ are each hydrogen, halo, or C₁-C₆ alkoxy and X is H, halo, nitro, C₁-C₆ alkyl, halo(C₁-C₆)-alkyl or C₁-C₆ alkoxy. Still more preferred are compounds wherein R₁, R₂, and R₃ are each hydrogen and X is H, halo or halo(C₁-C₆)-alkyl.

Preferred compounds of formula III include those wherein Y is CONR₇R₇′, NR₇CONR₇R₇′, or NR₇COR₇′. More preferred are compounds wherein Y is CONH₂, NHCONH₂, or NHCOR₇′. Even more preferred compounds of formula III are those wherein Y is NHCONH₂.

Preferred compounds of formula III also include those wherein R₄ is H or C₁-C₆ alkyl and R₅ is C₁-C₆ alkyl. Preferred compounds are also those wherein R₄ is C₁-C₆ alkyl and R₅ is C₁-C₆ alkyl.

Preferred compounds of formula III further include those wherein R₄ and R₅ together with the atoms to which they are attached form a 5-12 membered mono-, bi-, or tricyclic ring system, where the 5-12 membered ring is partially unsaturated, and wherein the 5-12 membered ring is optionally substituted with 1 or 2 R₂₂ groups. More preferred are compounds wherein R₄ and R₅ together with the atoms to which they are attached form a 6 membered ring, wherein the ring is optionally substituted with 1 or 2 R₂₂ groups.

Preferred compounds of formula III further include those wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, or C₃-C₇ cycloalkyl(C₁-C₆)alkyl. More preferred are compounds wherein R₆′ is H, halo, C₁-C₆ alkyl, halo(C₁-C₆)-alkyl, C₁-C₆ alkoxy, amino, or mono- or di-(C₁-C₆)alkylamino. Even more preferred are compounds wherein R₆′ is H, halo, or C₁-C₆ alkoxy.

Preferred compounds of formula III further include compounds of formula III-A: 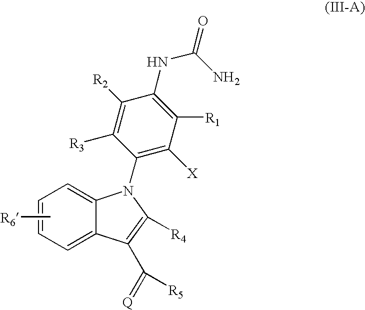
and pharmaceutically acceptable salts thereof, wherein
X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, mono C₃-C₆ alkenylamino, carboxamide, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
      where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
R₄ is H or C₁-C₆ alkyl optionally substituted with 1-2 groups selected from oxo, aryl, heteroaryl, or R₂₂;
R₅ is OR₇, NR₇R₇′, NR₇OR₇′, or C₁-C₆ alkyl optionally substituted with 1-2 groups selected from oxo, aryl, heteroaryl, or R₂₂; or
R₄ and R₅ together with the atoms to which they are attached form a 5-12 membered mono-, bi-, or tricyclic ring system, where the 5-12 membered ring is partially unsaturated and optionally contains one or two of oxygen, S(O)ₘ where m is 0, 1, or 2, nitrogen, NOR₇ or NR₇ where R₇ is hydrogen or C₁-C₆ alkyl, and wherein the 5-12 membered ring is optionally substituted with 1 or 2 R₂₂ groups;
R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups; and
Q is O or NOR₇.

Preferred compounds of formula III-A include those wherein X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferably, X, R₁, R₂, and R₃ are independently H, halo, C₁-C₆ alkyl, halo(C₁-C₆)-alkyl or C₁-C₆ alkoxy.

Preferred compounds of formula III-A also include those wherein R₁, R₂, and R₃ are each hydrogen. Further preferred are compounds wherein R₁, R₂, and R₃ are each hydrogen, halo, or C₁-C₆ alkoxy and X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferred are compounds wherein R₁, R₂, and R₃ are each hydrogen, halo, or C₁-C₆ alkoxy and X is H, halo, nitro, C₁-C₆ alkyl, halo(C₁-C₆)-alkyl or C₁-C₆ alkoxy. Still more preferred are compounds wherein R₁, R₂, and R₃ are each hydrogen and X is H, halo or halo(C₁-C₆)-alkyl.

Preferred compounds of formula III-A also include those wherein R₄ is H or C₁-C₆ alkyl and R₅ is C₁-C₆ alkyl. Preferred compounds are also those wherein R₄ is C₁-C₆ alkyl and R₅ is C₁-C₆ alkyl.

Preferred compounds of formula III-A further include those wherein R₄ and R₅ together with the atoms to which they are attached form a 5-12 membered mono-, bi-, or tricyclic ring system, where the 5-12 membered ring is partially unsaturated, and wherein the 5-12 membered ring is optionally substituted with 1 or 2 R₂₂ groups. More preferred are compounds wherein R₄ and R₅ together with the atoms to which they are attached form a 6 membered ring, wherein the ring is optionally substituted with 1 or 2 R₂₂ groups.

Preferred compounds of formula III-A further include those wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, or C₃-C₇ cycloalkyl(C₁-C₆)alkyl. More preferred are compounds wherein R₆′ is H, halo, C₁-C₆ alkyl, halo(C₁-C₆)-alkyl, C₁-C₆ alkoxy, amino, or mono- or di-(C₁-C₆)alkylamino. Even more preferred are compounds wherein R₆′ is H, halo, or C₁-C₆ alkoxy.

Preferred compounds of formula III-A further include those wherein Q is O or N—OH. More preferred are compounds where Q is O. Additionally preferred are compounds where Q is N—OH.

Other preferred compounds of formula III further include compounds of formula III-B: 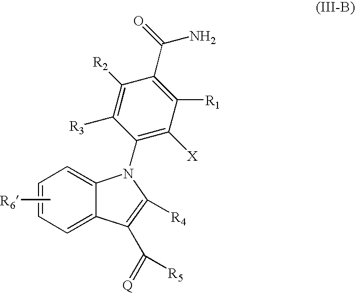
and pharmaceutically acceptable salts thereof, wherein
X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, mono C₃-C₆ alkenylamino, carboxamide, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
      where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
R₄ is H or C₁-C₆ alkyl optionally substituted with 1-2 groups selected from oxo, aryl, heteroaryl, or R₂₂;
R₅ is OR₇, NR₇R₇′, NR₇OR₇′, or C₁-C₆ alkyl optionally substituted with 1-2 groups selected from oxo, aryl, heteroaryl, or R₂₂; or
R₄ and R₅ together with the atoms to which they are attached form a 5-12 membered mono-, bi-, or tricyclic ring system, where the 5-12 membered ring is partially unsaturated and optionally contains one or two of oxygen, S(O)ₘ where m is 0, 1, or 2, nitrogen, NOR₇ or NR₇ where R₇ is hydrogen or C₁-C₆ alkyl, and wherein the 5-12 membered ring is optionally substituted with 1 or 2 R₂₂ groups;
R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups; and
Q is O or NOR₇.

Preferred compounds of formula III-B include those wherein X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferably, X, R₁, R₂, and R₃ are independently H, halo, C₁-C₆ alkyl, halo(C₁-C₆)-alkyl or C₁-C₆ alkoxy.

Preferred compounds of formula III-B also include those wherein R₁, R₂, and R₃ are each hydrogen. Further preferred are compounds wherein R₁, R₂, and R₃ are each hydrogen, halo, or C₁-C₆ alkoxy and X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferred are compounds wherein R₁, R₂, and R₃ are each hydrogen, halo, or C₁-C₆ alkoxy and X is H, halo, nitro, C₁-C₆ alkyl, halo(C₁-C₆)-alkyl or C₁-C₆ alkoxy. Still more preferred are compounds wherein R₁, R₂, and R₃ are each hydrogen and X is H, halo or halo(C₁-C₆)-alkyl.

Preferred compounds of formula III-B also include those wherein R₄ is H or C₁-C₆ alkyl and R₅ is C₁-C₆ alkyl. Preferred compounds are also those wherein R₄ is C₁-C₆ alkyl and R₅ is C₁-C₆ alkyl.

Preferred compounds of formula III-B further include those wherein R₄ and R₅ together with the atoms to which they are attached form a 5-12 membered mono-, bi-, or tricyclic ring system, where the 5-12 membered ring is partially unsaturated, and wherein the 5-12 membered ring is optionally substituted with 1 or 2 R₂₂ groups. More preferred are compounds wherein R₄ and R₅ together with the atoms to which they are attached form a 6 membered ring, wherein the ring is optionally substituted with 1 or 2 R₂₂ groups.

Preferred compounds of formula III-B further include those wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, or C₃-C₇ cycloalkyl(C₁-C₆)alkyl. More preferred are compounds wherein R₆′ is H, halo, C₁-C₆ alkyl, halo(C₁-C₆)-alkyl, C₁-C₆ alkoxy, amino, or mono- or di-(C₁-C₆)alkylamino. Even more preferred are compounds wherein R₆′ is H, halo, or C₁-C₆ alkoxy.

Preferred compounds of formula III-B further include those wherein Q is O or N—OH. More preferred are compounds where Q is O. Additionally preferred are compounds where Q is N—OH.

Preferred compounds of formulae I, II, and III further include compounds of formula IV: 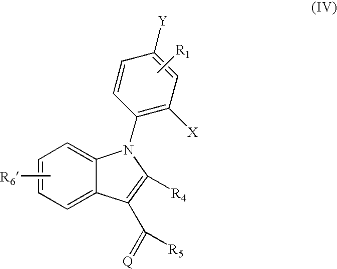
and pharmaceutically acceptable salts thereof, wherein
R₁ is H, halo, or C₁-C₆ alkoxy;
X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, mono C₃-C₆ alkenylamino, carboxamide, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
      where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
Y is H, NR₇R₇′, NR₇(C₃-C₆)alkenyl, NR₇CONHCOR₇′, —C(=Z)NR₇R₇′, —NR₇CONR₇R₇′, —NR₇COR₇′, or —NR₇—(C₁-C₆)alkyl-(C₁-C₆)alkoxy,
   where Z is O, S, or NOR₇, and
   R₇ and R₇′ at each occurrence are independently selected from H and C₁-C₆ alkyl;
R₄ is H or C₁-C₆ alkyl optionally substituted with 1-2 groups selected from oxo, aryl, heteroaryl, or R₂₂;
R₅ is OR₇, NR₇R₇′, NR₇OR₇′, or C₁-C₆ alkyl optionally substituted with 1-2 groups selected from oxo, aryl, heteroaryl, or R₂₂; or
R₄ and R₅ together with the atoms to which they are attached form a 5-12 membered mono-, bi-, or tricyclic ring system, where the 5-12 membered ring is partially unsaturated and optionally contains one or two of oxygen, S(O)ₘ where m is 0, 1, or 2, nitrogen, NOR₇ or NR₇ where R₇ is hydrogen or C₁-C₆ alkyl, and wherein the 5-12 membered ring is optionally substituted with 1 or 2 R₂₂ groups;
R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups; and
Q is ═O or ═NOR₇.

Preferred compounds of formula IV include those wherein X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferably, X is H, halo, C₁-C₆ alkyl, halo(C₁-C₆)-alkyl or C₁-C₆ alkoxy.

Preferred compounds of formula IV also include those wherein R₁ is hydrogen, halo, or C₁-C₆ alkoxy. Further preferred are compounds wherein R₁ is hydrogen, halo, or C₁-C₆ alkoxy and X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferred are compounds wherein R₁ is hydrogen and X is H, halo, nitro, C₁-C₆ alkyl, halo(C₁-C₆)-alkyl or C₁-C₆ alkoxy. Still more preferred are compounds wherein R₁ is hydrogen and X is H, halo or halo(C₁-C₆)-alkyl.

Preferred compounds of formula IV include those wherein Y is CONR₇R₇′, NR₇CONR₇R₇′, or NR₇COR₇′. More preferred are compounds wherein Y is CONH₂, NHCONH₂, or NHCOR₇′. Even more preferred compounds of formula IV are those wherein Y is NHCONH₂.

Preferred compounds of formula IV also include those wherein R₄ is H or C₁-C₆ alkyl and R₅ is C₁-C₆ alkyl. Preferred compounds are also those wherein R₄ is C₁-C₆ alkyl and R₅ is C₁-C₆ alkyl.

Preferred compounds of formula IV further include those wherein R₄ and R₅ together with the atoms to which they are attached form a 5-12 membered mono-, bi-, or tricyclic ring system, where the 5-12 membered ring is partially unsaturated, and wherein the 5-12 membered ring is optionally substituted with 1 or 2 R₂₂ groups. More preferred are compounds wherein R₄ and R₅ together with the atoms to which they are attached form a 6 membered ring, wherein the ring is optionally substituted with 1 or 2 R₂₂ groups.

Preferred compounds of formula IV further include those wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, or C₃-C₇ cycloalkyl(C₁-C₆)alkyl. More preferred are compounds wherein R₆′ is H, halo, C₁-C₆ alkyl, halo(C₁-C₆)-alkyl, C₁-C₆ alkoxy, amino, or mono- or di-(C₁-C₆)alkylamino. Even more preferred are compounds wherein R₆′ is H or halo.

Preferred compounds of formula I further include compounds of formula V: 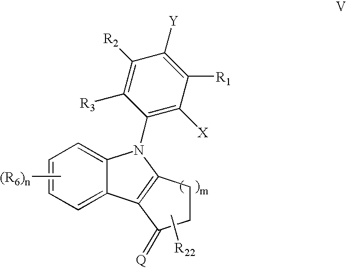
and the pharmaceutically acceptable salts thereof, wherein
X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, mono C₃-C₆ alkenylamino, carboxamide, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
      where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
or R₂ and R₃ together with the atoms to which they are attached form a 5-12 membered mono-, bi-, or tricyclic ring system, where the 5-12 membered ring is partially unsaturated or aromatic and optionally contains one or two of oxygen, S(O)ₘ where m is 0, 1, or 2, nitrogen, or NR₇ where R₇ is hydrogen or C₁-C₆ alkyl, and wherein the 5-12 membered ring is optionally substituted with 1 or 2 R₂₂ groups;
or R₁ and X together with the atoms to which they are attached form a 5-12 membered mono-, bi-, or tricyclic ring system, where the 5-12 membered ring is partially unsaturated or aromatic and optionally contains one or two of oxygen, S(O)ₘ where m is 0, 1, or 2, nitrogen, or NR₇ where R₇ is hydrogen or C₁-C₆ alkyl, and wherein the 5-12 membered ring is optionally substituted with 1 or 2 R₂₂ groups;
Y is H, NR₇R₇′, NR₇(C₃-C₆)alkenyl, NR₇CONHCOR₇′, C(=Z)NR₇R₇′, NR₇CONR₇R₇′, NR₇COR₇′, or —NR₇—(C₁-C₆)alkyl-(C₁-C₆)alkoxy,
   where Z is O, S, or NOR₇, and
   R₇ and R₇′ at each occurrence are independently selected from H and C₁-C₆ alkyl;
m is 1, 2, or 3;
n is 0, 1, 2, 3, or 4;
R₆ at each occurrence is independently halo, C₁-C₆ alkyl, halo (C₁-C₆)-alkyl, C₁-C₆ alkoxy, nitro, hydroxyl, cyano, alkenyl, alkynyl, thiol, amino, mono- or di-(C₁-C₆) alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl groups and heteroaryl groups is optionally substituted with from 1-4 R₂₂ groups;
or two R₆ groups on adjacent carbons, together with the atoms to which they are attached, form a 5-12 membered mono-, bi-, or tricyclic ring system fused to the ring to which Y is attached, where the 5-12 membered ring is partially unsaturated or aromatic and optionally contains one or two of oxygen, S(O)ₘ where m is 0, 1, or 2, nitrogen, or NR₇ where R₇ is hydrogen or C₁-C₆ alkyl, wherein the 5-12 membered ring is optionally substituted with 1 or 2 R₂₂ groups; and
Q is O or NOR₇.

Preferred compounds of Formula V include those wherein X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl or heteroaryl, where each of the foregoing aryl groups and heteroaryl groups is optionally substituted with from 1-4 R₂₂ groups. Also preferred are compounds wherein R₁, R₂, and R₃ are each hydrogen, halo, or C₁-C₆ alkoxy. Further preferred are compounds wherein X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferred are compounds wherein R₁, R₂, and R₃ are each hydrogen, halo, or C₁-C₆ alkoxy and X is H, halo, C₁-C₆ alkyl, halo(C₁-C₆)-alkyl or C₁-C₆ alkoxy. Still more preferred are compounds wherein R₁, R₂, and R₃ are each hydrogen and X is halo.

Preferred compounds of formula V also include those wherein m is 2 (i.e., the ring to which Q is attached is 6 membered).

Preferred compounds of formula V are also those wherein n is 0.

Preferred compounds of formula V further include those wherein n is 1 and R₆ is halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl or heteroaryl, where each of the foregoing aryl groups and heteroaryl groups is optionally substituted with from 1-4 R₂₂ groups.

Preferred compounds of formula V further include compounds of formula VI: 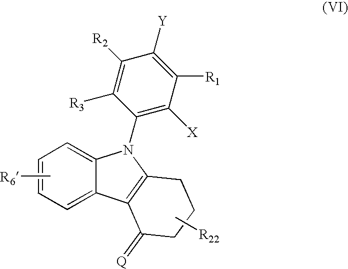
and pharmaceutically acceptable salts thereof, wherein
X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, mono C₃-C₆ alkenylamino, carboxamide, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
      where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
Y is H, NR₇R₇′, NR₇(C₃-C₆)alkenyl, NR₇CONHCOR₇′, —C(=Z)NR₇R₇′, —NR₇CONR₇R₇′, —NR₇COR₇′, or —NR₇—(C₁-C₆)alkyl-(C₁-C₆)alkoxy,
   where Z is O, S, or NOR₇, and
   R₇ and R₇′ at each occurrence are independently selected from H and C₁-C₆ alkyl;
R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups; and
Q is O or NOR₇.

Preferred compounds of formula VI include those wherein X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferably, X, R₁, R₂, and R₃ are independently H, halo, C₁-C₆ alkyl, halo(C₁-C₆)-alkyl or C₁-C₆ alkoxy.

Preferred compounds of formula VI also include those wherein R₁, R₂, and R₃ are each hydrogen, halo, or C₁-C₆ alkoxy. Further preferred are compounds wherein R₁, R₂, and R₃ are each hydrogen, halo, or C₁-C₆ alkoxy and X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆) alkylamino. More preferred are compounds wherein R₁, R₂, and R₃ are each hydrogen and X is H, halo, nitro, C₁-C₆ alkyl, halo (C₁-C₆)-alkyl or C₁-C₆ alkoxy. Still more preferred are compounds wherein R₁, R₂, and R₃ are each hydrogen and X is H, halo or halo(C₁-C₆)-alkyl.

Preferred compounds of formula VI include those wherein Y is CONR₇R₇′, NR₇CONR₇R₇′, or NR₇COR₇′. More preferred are compounds wherein Y is CONH₂, NHCONH₂, or NHCOR₇′. Even more preferred compounds of formula VI are those wherein Y is NHCONH₂.

Preferred compounds of formula VI further include those wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, or C₃-C₇ cycloalkyl(C₁-C₆)alkyl. More preferred are compounds wherein R₆′ is H, halo, C₁-C₆ alkyl, halo(C₁-C₆)-alkyl, C₁-C₆ alkoxy, amino, or mono- or di-(C₁-C₆)alkylamino. Even more preferred are compounds wherein R₆′ is H or halo.

Preferred compounds of formula VI also include those wherein R₂₂ on the ring to which Q is attached is hydrogen.

Preferred compounds of formula VI also include those wherein Q is O.

Preferred compounds are also those wherein Q is NOR₇. More preferably, Q is NOH.

Preferred compounds of formula VI further include compounds of formula VI-A: 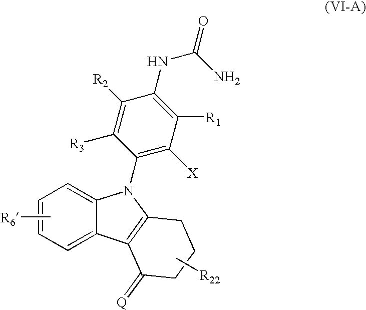
and pharmaceutically acceptable salts thereof, wherein
X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
      where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups; and Q is O or NOR₇, where R₇ is H or C₁-C₆ alkyl.

Preferred compounds of formula VI-A include those wherein X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferably, X, R₁, R₂, and R₃ are independently H, halo, C₁-C₆ alkyl, halo(C₁-C₆)-alkyl or C₁-C₆ alkoxy.

Preferred compounds of formula VI-A also include those wherein R₁, R₂, and R₃ are each hydrogen, halo, or C₁-C₆ alkoxy. Further preferred are compounds wherein R₁, R₂, and R₃ are each hydrogen, halo, or C₁-C₆ alkoxy and X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆) alkylamino. More preferred are compounds wherein R₁, R₂, and R₃ are each hydrogen, halo, or C₁-C₆ alkoxy and X is H, halo, nitro, C₁-C₆ alkyl, halo(C₁-C₆)-alkyl or C₁-C₆ alkoxy. Still more preferred are compounds wherein R₁, R₂, and R₃ are each hydrogen and X is H, halo or halo(C₁-C₆)-alkyl.

Preferred compounds of formula VI-A further include those wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, or C₃-C₇ cycloalkyl(C₁-C₆)alkyl. More preferred are compounds wherein R₆′ is H, halo, C₁-C₆ alkyl, halo(C₁-C₆)-alkyl, C₁-C₆ alkoxy, amino, or mono- or di-(C₁-C₆)alkylamino. Even more preferred are compounds wherein R₆′ is H, C₁-C₆ alkoxy, or halo.

Preferred compounds of formula VI-A also include those wherein R₂₂ on the ring to which Q is attached is hydrogen.

Preferred compounds of formula VI-A also include those wherein Q is O.

Preferred compounds are also those wherein Q is NOR₇. More preferably, Q is NOH.

Preferred compounds of formula VI further include compounds of formula VI-B: 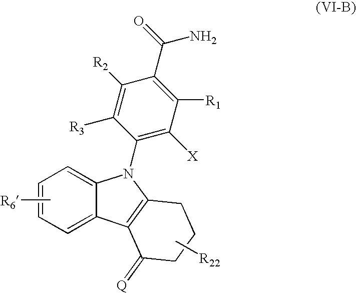
and pharmaceutically acceptable salts thereof, wherein
X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, mono C₃-C₆ alkenylamino, carboxamide, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
      where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups; and
Q is O or NOR₇.

Preferred compounds of formula VI-B include those wherein X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferably, X, R₁, R₂, and R₃ are independently H, halo, C₁-C₆ alkyl, halo(C₁-C₆)-alkyl or C₁-C₆ alkoxy.

Preferred compounds of formula VI-B also include those wherein R₁, R₂, and R₃ are each hydrogen, halo, or C₁-C₆ alkoxy. Further preferred are compounds wherein R₁, R₂, and R₃ are each hydrogen, halo, or C₁-C₆ alkoxy and X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆) alkylamino. More preferred are compounds wherein R₁, R₂, and R₃ are each hydrogen, halo, or C₁-C₆ alkoxy and X is H, halo, nitro, C₁-C₆ alkyl, halo(C₁-C₆)-alkyl or C₁-C₆ alkoxy. Still more preferred are compounds wherein R₁, R₂, and R₃ are each hydrogen and X is H, halo or halo(C₁-C₆)-alkyl.

Preferred compounds of formula VI-B further include those wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, or C₃-C₇ cycloalkyl(C₁-C₆)alkyl. More preferred are compounds wherein R₆′ is H, halo, C₁-C₆ alkyl, halo(C₁-C₆)-alkyl, C₁-C₆ alkoxy, amino, or mono- or di-(C₁-C₆)alkylamino. Even more preferred are compounds wherein R₆′ is H, C₁-C₆ alkoxy, or halo.

Preferred compounds of formula VI-B also include those wherein R₂₂ on the ring to which Q is attached is hydrogen.

Preferred compounds of formula VI-B also include those wherein Q is O.

Preferred compounds of formula VI-B are also those wherein Q is NOR₇. More preferably, Q is NOH.

Preferred compounds of formula I further include compounds of formula VII: 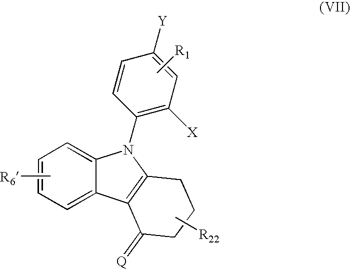
and pharmaceutically acceptable salts thereof, wherein
R₁ is H, C₁-C₆ alkoxy, or halo;
X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups;
      where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
Y is H, NR₇R₇′, NR₇(C₃-C₆)alkenyl, NR₇CONHCOR₇′, —C(=Z)NR₇R₇′, —NR₇CONR₇R₇′, —NR₇COR₇′, or —NR₇—(C₁-C₆)alkyl-(C₁-C₆)alkoxy,
   where Z is O, S, or NOR₇, and
   R₇ and R₇′ at each occurrence are independently selected from H and C₁-C₆ alkyl;
R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
   where each of the foregoing aryl and heteroaryl groups is optionally substituted with from 1-4 R₂₂ groups; and
Q is ═O or ═NOR₇.

Preferred compounds of formula VII include those wherein X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferably, X is H, halo, C₁-C₆ alkyl, halo(C₁-C₆)-alkyl or C₁-C₆ alkoxy.

Preferred compounds of formula VII also include those wherein R₁ is hydrogen, halo, or C₁-C₆ alkoxy. Further preferred are compounds wherein R₁ is hydrogen, halo, or C₁-C₆ alkoxy and X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino. More preferred are compounds wherein R₁ is hydrogen, halo, or C₁-C₆ alkoxy and X is H, halo, nitro, C₁-C₆ alkyl, halo(C₁-C₆)-alkyl or C₁-C₆ alkoxy. Still more preferred are compounds wherein R₁ is hydrogen and X is H, halo or halo(C₁-C₆)-alkyl.

Preferred compounds of formula VII include those wherein Y is CONR₇R₇′, NR₇CONR₇R₇′, or NR₇COR₇′. More preferred are compounds wherein Y is CONH₂, NHCONH₂, or NHCOR₇′. Even more preferred compounds of formula VII are those wherein Y is NHCONH₂.

Preferred compounds of formula VII further include those wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, or C₃-C₇ cycloalkyl(C₁-C₆)alkyl. More preferred are compounds wherein R₆′ is H, halo, C₁-C₆ alkyl, halo(C₁-C₆)-alkyl, C₁-C₆ alkoxy, amino, or mono- or di-(C₁-C₆)alkylamino. Even more preferred are compounds wherein R₆′ is H or halo.

Preferred compounds of formula VII also include those wherein R₂₂ on the ring to which Q is attached is hydrogen.

Preferred compounds of formula VII also include those wherein Q is O.

Preferred compounds are also those wherein Q is NOR₇. More preferably, Q is NOH.

Preferred compounds of formula I further include compounds of formula VIII: 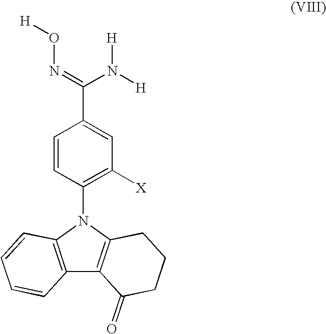
and pharmaceutically acceptable salts thereof, wherein X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino. More preferably, X is H, halo, C₁-C₆ alkyl, halo(C₁-C₆)-alkyl or C₁-C₆ alkoxy. Still more preferably, X is H, halo or halo(C₁-C₆)-alkyl.

Preferred compounds of formula I further include compounds of formula IX: 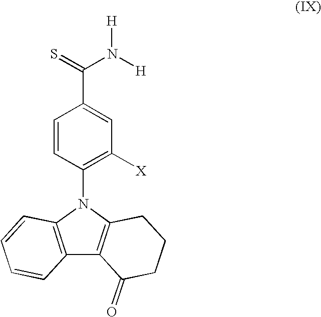
and pharmaceutically acceptable salts thereof, wherein X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino. More preferably, X is H, halo, C₁-C₆ alkyl, halo(C₁-C₆)-alkyl or C₁-C₆ alkoxy. Still more preferably, X is H, halo or halo(C₁-C₆)-alkyl.

Preferred compounds of formula I further include compounds of formula X: 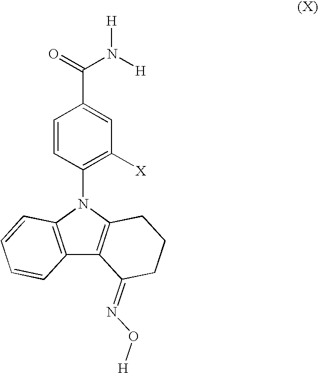
and pharmaceutically acceptable salts thereof, wherein X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino. More preferably, X is H, halo, C₁-C₆ alkyl, halo(C₁-C₆)-alkyl or C₁-C₆ alkoxy. Still more preferably, X is H, halo or halo(C₁-C₆)-alkyl.

Preferred compounds of formula I further include compounds of formula XI: 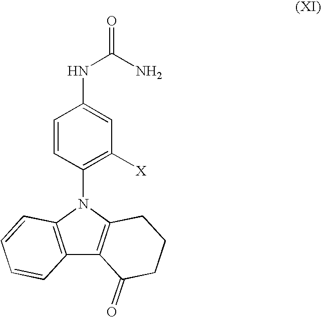
and pharmaceutically acceptable salts thereof, wherein X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino. More preferably, X is H, halo, C₁-C₆ alkyl, halo(C₁-C₆)-alkyl or C₁-C₆ alkoxy. Still more preferably, X is H, halo or halo(C₁-C₆)-alkyl.

In another aspect, the invention encompasses compositions comprising a compound or pharmaceutically acceptable salt of Formula I and at least one pharmaceutically acceptable carrier, solvent, adjuvant or diluent.

In another aspect, the invention encompasses a method of treating diseases and/or conditions related to cell proliferation and/or abnormal cell mitosis, such as cancer, inflammation and inflammation-associated disorders, and conditions associated with angiogenesis, the method comprising administering to a patient in need of such treatment, a pharmaceutically acceptable amount of a compound or salt of Formula I or a pharmaceutical composition comprising a compound or salt of Formula I.

In a preferred embodiment, the invention relates to a method for treating disease states characterized by abnormal cell mitosis, the method comprising administering to a patient in need of such treatment, a pharmaceutically acceptable amount of a compound or salt of Formula I or a pharmaceutical composition comprising a compound or salt of Formula I.

In a further preferred embodiment, the invention provides a method for inhibiting tubulin polymerization, inhibiting tumor development, inhibiting the rate of tumor growth, and/or inducing regression of pre-existing tumors comprising administering to a patient an effective amount of a compound of formula I or salt thereof, or a composition comprising a compound of formula I or salt thereof.

The term “alkoxy” represents an alkyl group of indicated number of carbon atoms attached to the parent molecular moiety through an oxygen bridge. Examples of alkoxy groups include, for example, methoxy, ethoxy, propoxy and isopropoxy.

As used herein, the term “alkyl” includes those alkyl groups of a designed number of carbon atoms. Alkyl groups may be straight, or branched. Examples of “alkyl” include methyl, ethyl, propyl, isopropyl, butyl, iso-, sec- and tert-butyl, pentyl, hexyl, heptyl, 3-ethylbutyl, and the like.

The term “aryl” refers to an aromatic hydrocarbon ring system containing at least one aromatic ring. The aromatic ring may optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl, naphthyl, and anthracenyl. More preferred aryl groups are phenyl and naphthyl. Most preferred is phenyl. The aryl groups of the invention may be substituted with various groups as provided herein. Thus, any carbon atom present within an aryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The term “cycloalkyl” refers to a C₃-C₈ cyclic hydrocarbon. Examples of cycloalkyl include cyclopropyl, cyclobutyl, cyclopentyl, cyclohexyl, cycloheptyl and cyclooctyl. More preferred are C₃-C₆ cycloalkyl groups. The cycloalkyl groups of the invention may be substituted with various groups as provided herein. Thus, any carbon atom present within a cycloalkyl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The terms “halogen” or “halo” indicate fluorine, chlorine, bromine, and iodine.

The term “haloalkoxy” refers to an alkoxy group substituted with one or more halogen atoms, where each halogen is independently F, Cl, Br or I. Preferred halogens are F and Cl. Preferred haloalkoxy groups contain 1-6 carbons, more preferably 1-4 carbons, and still more preferably 1-2 carbons. “Haloalkoxy” includes perhaloalkoxy groups, such as OCF₃ or OCF₂CF₃. A preferred haloalkoxy group is trifluoromethoxy.

The term “haloalkyl” refers to an alkyl group substituted with one or more halogen atoms, where each halogen is independently F, Cl, Br or I. Preferred halogens are F and Cl. Preferred haloalkyl groups contain 1-6 carbons, more preferably 1-4 carbons, and still more preferably 1-2 carbons. “Haloalkyl” includes perhaloalkyl groups, such as CF₃ or CF₂CF₃. A preferred haloalkyl group is trifluoromethyl.

The term “heterocycloalkyl” refers to a ring or ring system containing at least one heteroatom selected from nitrogen, oxygen, and sulfur, wherein said heteroatom is in a non-aromatic ring. The heterocycloalkyl ring is optionally fused to or otherwise attached to other heterocycloalkyl rings and/or non-aromatic hydrocarbon rings and/or phenyl rings. Preferred heterocycloalkyl groups have from 3 to 7 members. More preferred heterocycloalkyl groups have 5 or 6 members. Examples of heterocycloalkyl groups include, for example, 1,2,3,4-tetrahydroisoquinolinyl, piperazinyl, morpholinyl, piperidinyl, tetrahydrofuranyl, pyrrolidinyl, pyridinonyl, and pyrazolidinyl. Preferred heterocycloalkyl groups include piperidinyl, piperazinyl, morpholinyl, pyrrolidinyl, pyridinonyl, dihydropyrrolidinyl, and pyrrolidinonyl. The heterocycloalkyl groups of the invention may be substituted with various groups as provided herein. Thus, any atom present within a heterocycloalkyl ring and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The term “heteroaryl” refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Examples of heteroaryl groups include, for example, pyridine, furan, thienyl, 5,6,7,8-tetrahydroisoquinoline and pyrimidines. The heteroaryl groups of the invention may be substituted with various groups as provided herein. Thus, any carbon atom present within an heteroaryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

Preferred examples of heteroaryl groups include thienyl, benzothienyl, pyridyl, quinolyl, pyrazolyl, pyrimidyl, imidazolyl, benzimidazolyl, furanyl, benzofuranyl, dibenzofuranyl, thiazolyl, benzothiazolyl, isoxazolyl, oxadiazolyl, isothiazolyl, benzisothiazolyl, triazolyl, pyrrolyl, indolyl, pyrazolyl, and benzopyrazolyl.

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

The compounds of general Formula I may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants, and if desired other active ingredients. The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of general Formula I may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For disorders of the eye or other external tissues, e.g., mouth and skin, the formulations are preferably applied as a topical gel, spray, ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The antiinflammatory active ingredients are preferably present in such formulations in a concentration of 0.5 to 20%, advantageously 0.5 to 10% and particularly about 1.5% w/w. For therapeutic purposes, the active compounds of this combination invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water. Preferred non-human animals include domesticated animals.

The compounds of the present invention may be prepared by use of known chemical reactions and procedures. Representative methods for synthesizing compounds of the invention are presented below. It is understood that the nature of the substituents required for the desired target compound often determines the preferred method of synthesis. All variable groups of these methods are as described in the generic description if they are not specifically defined below.

Methods of Preparation

General Procedure

Representative synthetic procedures for the preparation of compounds of the invention are outlined in the schemes below.

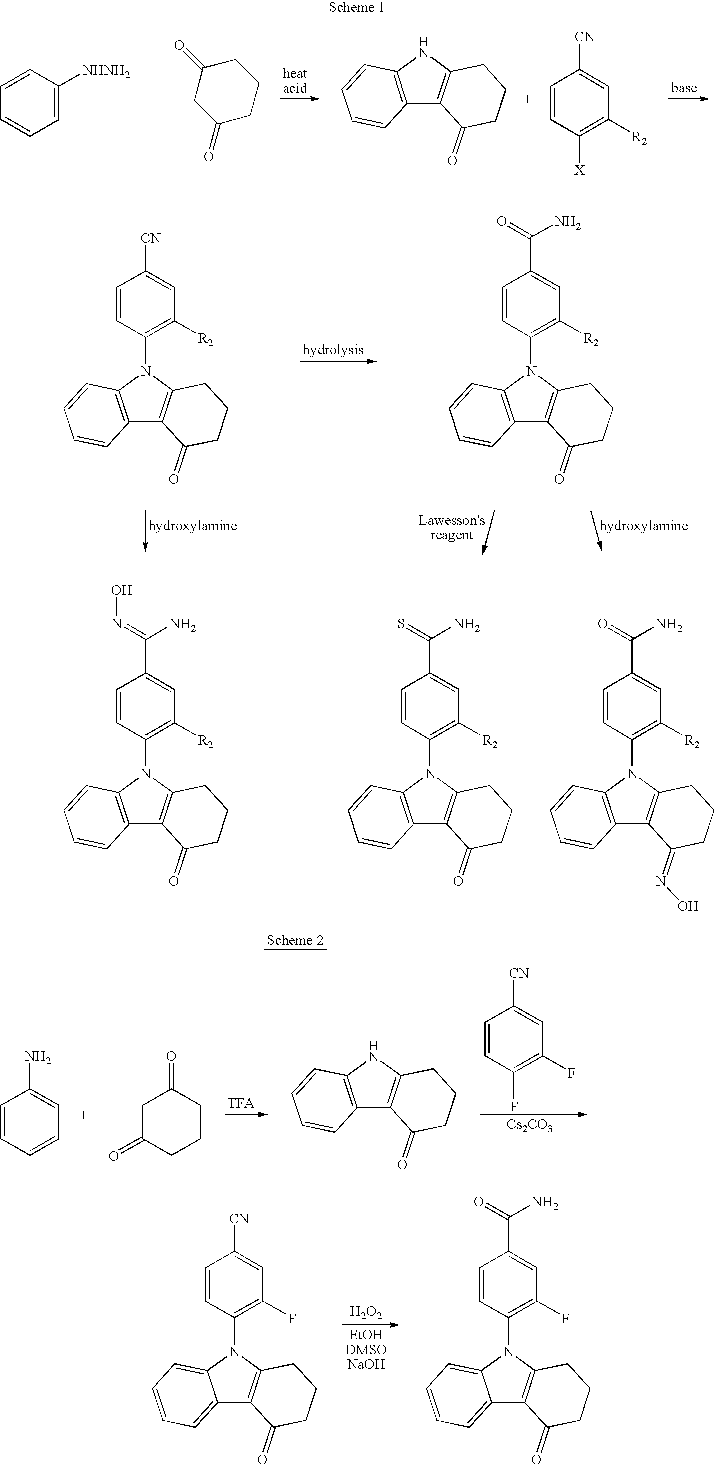

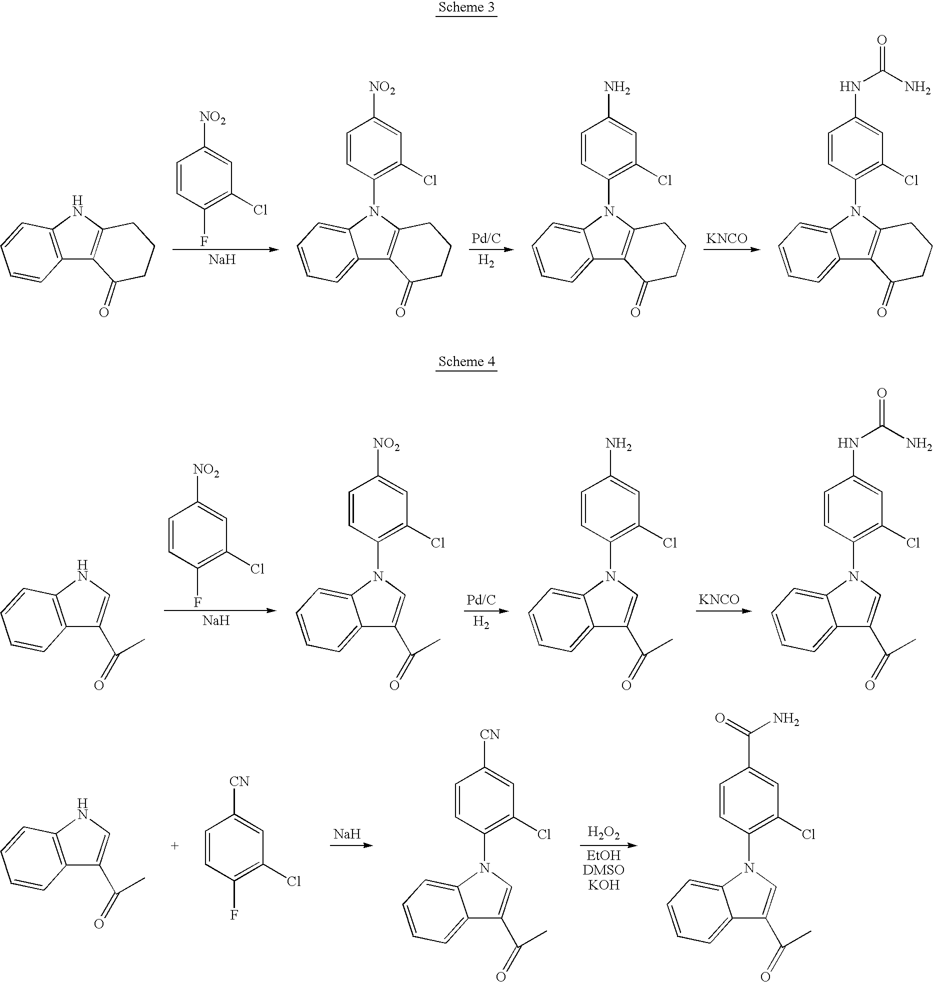

Those having skill in the art will recognize that the starting materials and reaction conditions may be varied, the sequence of the reactions altered, and additional steps employed to produce compounds encompassed by the present invention, as demonstrated by the following examples. In some cases, protection of certain reactive functionalities may be necessary to achieve some of the above transformations. In general, the need for such protecting groups as well as the conditions necessary to attach and remove such groups will be apparent to those skilled in the art of organic synthesis.

The disclosures of all articles and references mentioned in this application, including patents, are incorporated herein by reference in their entirety.

Structures were named using ChemDraw version 8.0, which is available from Cambridgesoft.com in Cambridge, Mass.

### EXAMPLES

The preparation of intermediates and compounds of the invention is illustrated further by the following examples, which are not to be construed as limiting the invention in scope or spirit to the specific procedures and compounds described in them. In all cases, unless otherwise specified, the column chromatography is performed using a silica gel solid phase.

### Example 1

### 3-Bromo-4-(4-oxo-1,2,3,4-tetrahydro-carbazol-9-yl)benzamide (Compound 1)

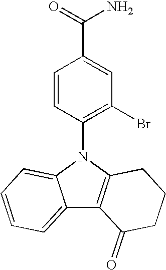

Trifluoroacetic acid (6 mL) is added to 1,3-cyclohexanedione (24 mmol, 2.69 g) in a 10-20 mL microwave reactor. The reactor is cooled in an ice bath, and phenylhydrazine (20 mmol, 1.96 mL) is added. The mixture is stirred for 5 minutes, then sealed and heated using a Personal Chemistry microwave apparatus set to 140 degrees Celsius at very high absorbance for 600 seconds. Upon cooling, the crude mixture is extracted using methylene chloride (250 mL) and saturated sodium hydrogen carbonate (100 mL). The organic layer is dried over magnesium sulfate. Concentration and chromatography to afford the desired 1,2,3,9-Tetrahydro-carbazole-4-one as a brown solid (1.27 g, 34%).

Sodium Hydride (60% oil suspension, 12 mmol, 0.48 g) is triturated with hexane and suspended in N,N-dimethylformamide (6 mL). 1,2,3,9-Tetrahydro-carbazole-4-one (6 mmol, 1.11 g) is added in several portions to the water-cooled suspension. After 5 minutes, 3-bromo,4-fluorobenzonitrile (8.5 mmol, 1.7 g) is added. The reaction is stirred at ambient temperature for 50 minutes, then at 50 C for 30 minutes. The reaction mixture is allowed to cool and is extracted into ethyl acetate (400 mL) and is washed with water (100 mL). The organic phase is dried over magnesium sulfate. Filtration, followed by concentration, silica gel chromatography, and trituration with ethyl acetate to afford the desired 3-Bromo-4-(4-oxo-1,2,3,4-tetrahydro-carbazol-9-yl)benzonitrile as a tan solid (951 mg, 43%).

To the 3-Bromo-4-(4-oxo-1,2,3,4-tetrahydro-carbazol-9-yl)benzonitrile (2.2 mmol, 800 mg) is added DMSO (0.2 mL), abs. ethanol (15 mL), and KOH (840 mg). The mixture is loared into a 40 degree Celsius oil bath, and 30% hydrogen peroxide (4 mL) is added. After 20 minutes, the reaction is taken up in ethyl acetate (400 mL) and washed with water (100 mL). The organic phase is dried using magnesium sulfate, filtered, concentrated, and chromatographed. The residue is triturated with ethyl acetate and dried in vacuo, affording 401 mg of the desired 3-Bromo-4-(4-oxo-1,2,3,4-tetrahydro-carbazol-9-yl)benzamide as a white solid (52%). LCMS M+H=383.

### Example 2

### 3-Chloro-4-(4-oxo-1,2,3,4-tetrahydro-carbazol-9-yl)benzamide (Compound 2)

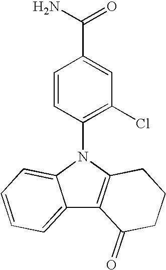

Sodium Hydride (60% oil suspension, 42 mmol, 1.68 g) is triturated with hexane and suspended in N,N-dimethylformamide (25 mL). 1,2,3,9-Tetrahydro-carbazole-4-one (21 mmol, 3.90 g) is added in several portions to the water-cooled suspension. After 5 minutes, 3-chloro,4-fluorobenzonitrile (28 mmol, 4.35 g) is added, and the flask is lowered into a 50 degree Celsius oil bath. After 1 hour, the reaction mixture is allowed to cool and is extracted into ethyl acetate (1 L) and is washed with water (200 mL). The organic phase is dried over magnesium sulfate. Filtration, followed by concentration and silica gel chromatography (hexane:ethyl acetate 1:1) affords the desired 3-Chloro-4-(4-oxo-1,2,3,4-tetrahydro-carbazol-9-yl)benzonitrile as a solid (3.97 g, 59%)

To 3-Chloro-4-(4-oxo-1,2,3,4-tetrahydro-carbazol-9-yl)benzonitrile (12 mmol, 3.97 g) is added DMSO (1 mL), abs. ethanol (40 mL), and KOH (2.88 g). The mixture is loared into a 50 degree Celsius oil bath, and 30% hydrogen peroxide (6 mL) is added. After 15 minutes, the reaction is taken up in ethyl acetate (900 mL) and washed with water (250 mL). The organic phase is dried using magnesium sulfate, then filtered, concentrated and chromatographed on 120 g silica eluting with ethyl acetate, affording 1.85 g of the desired 3-Chloro-4-(4-oxo-1,2,3,4-tetrahydro-carbazol-9-yl)benzamide as a foam (41%). LCMS M+H=339.

### Example 3

### 3-Bromo-N-hydroxy-4-(4-oxo-1,2,3,4-tetrahydro-carbazol-9-yl)-benzamidine (Compound 3)

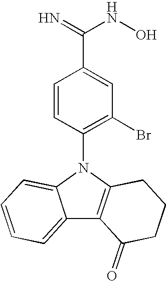 3-Bromo-4-(4-oxo-1,2,3,4-tetrahydro-carbazol-9-yl)benzonitrile (0.41 mmol, 150 mg) is combined with hydroxylamine hydrochloride (217 mg). Methanol (2 mL) and triethylamine (0.5 mL) are added. The flask is stoppered and stirred at ambient temperature for 16 h. Concentration followed by silica gel chromatography affords the desired 3-Bromo-N-hydroxy-4-(4-oxo-1,2,3,4-tetrahydro-carbazol-9-yl)-benzamidine as a waxy solid (63 mg, 39%). LCMS M+H=398.

### Example 4

### 3-Bromo-4-(4-hydroxyimino-1,2,3,4-tetrahydro-carbazol-9-yl)benzamide (Compound 4)

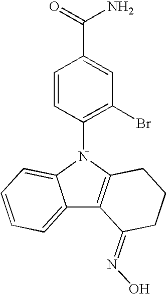

3-Bromo-4-(4-oxo-1,2,3,4-tetrahydro-carbazol-9-yl)benzamide (0.096 mmol, 35 mg), hydroxylamine hydrochloride (35 mg), methanol (0.2 mL) and triethylamine (0.2 mL) are combined and stirred, stoppered, at 45 degrees Celsius for 22 h. Concentration followed by chromatography affords the desired 3-Bromo-4-(4-hydroxyimino-1,2,3,4-tetrahydro-carbazol-9-yl)benzamide as a white solid (19 mg, 50%). LCMS M+H=398.

### Example 5

### 3-Bromo-4-(4-oxo-1,2,3,4-tetrahydro-carbazol-9-yl)thiobenzamide (Compound 5)

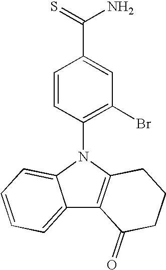

To 3-Bromo-4-(4-oxo-1,2,3,4-tetrahydro-carbazol-9-yl)benzamide (0.13 mmol, 51 mg) is added Lawesson's reagent (0.07 mmol, 28 mg) and toluene (0.6 mL). The mixture is brought to reflux for 15 minutes, then allowed to cool to ambient. The reaction is diluted with ethyl acetate (50 mL) and washed with saturated sodium bicarbonate (20 mL). The organic layer is dried over magnesium sulfate, concentrated, and subjected to chromatography, affording the desired 3-Bromo-4-(4-oxo-1,2,3,4-tetrahydro-carbazol-9-yl)thiobenzamide as an orange solid (10 mg, 19%). LCMS M+H=399.

### Example 6

### 4-(3-Acetyl-indol-1-yl)-3-chloro-benzamide (Compound 6)

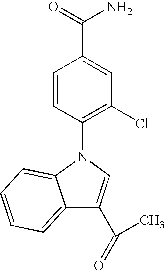

Sodium Hydride (60% oil suspension, 4 mmol, 0.160 mg) is triturated with hexane and suspended in N,N-dimethylformamide (3 mL). 3-Acetylindole (2 mmol, 318 mg) is added to the water-cooled suspension. After 5 minutes, 3-chloro,4-fluorobenzonitrile (3 mmol, 368 mg) is added. The reaction is stirred at 50 degrees Celsius for 45 minutes. The reaction mixture is allowed to cool and is extracted into ethyl acetate (200 mL) and is washed with water (50 mL). The organic phase is dried over magnesium sulfate. Filtration, followed by concentration, and silica gel chromatography affords the desired 4-(3-Acetyl-indol-1-yl)-3-chloro-benzonitrile as a tan solid (309 mg, 52%)

4-(3-Acetyl-indol-1-yl)-3-chloro-benzonitrile (0.90 mmol, 283 mg) is hydrolyzed by the method of example 1, using DMSO (10 drops), abs. ethanol (3 mL), KOH (209 mg), and 30% hydrogen peroxide (ca. 2 mL). Upon workup and chromatography the desired 4-(3-Acetyl-indol-1-yl)-3-chloro-benzamide is obtained as a yellow solid (263 mg, 93%). LCMS M+H=313.

### Example 7

### 4-(3-(1-hydroxyimino-ethyl)-indol-1-yl)-3-chloro-benzamide (Compound 7)

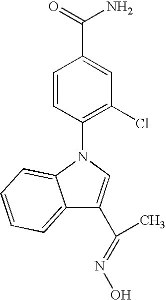

4-(3-Acetyl-indol-1-yl)-3-chloro-benzamide (0.12 mmol, 37 mg) is combined with hydroxylamine hydrochloride (74 mg), triethylamine (1 mL) and methanol (1 mL). The flask is stirred, stoppered, at ambient for 16 h; at 50 degrees Celsius for 8 h; and at ambient for and additional 16 hours. Concentration is followed by extraction using ethyl acetate (100 mL)/water (50 mL). The organic layer is dried over magnesium sulfate, filtered through 5-10 gm silica gel, and concentrated to afford the desired 4-(3-(1-hydroxyimino-ethyl)-indol-1-yl)-3-chloro-benzamide (33 mg, 83%) as a white foam. LCMS M+H=328.

### Example 8

### 4-(3-Acetyl-indol-1-yl)-2-bromo-benzamide (Compound 8)

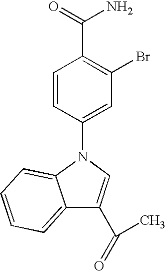

Sodium Hydride (60% oil suspension, 20 mmol, 800 mg) is triturated with hexane and suspended in N,N-dimethylformamide (10 mL). 3-Acetylindole (10 mmol, 1.59 g) is added to the ice-cooled suspension. After 5 minutes, 2-bromo, 4-fluorobenzonitrile (14 mmol, 2.8 g) is added. The reaction is stirred at 50 degrees Celsius for 30 minutes. The reaction mixture is allowed to cool and is extracted into ethyl acetate (200 mL) and is washed with water (50 mL). The organic phase is dried over magnesium sulfate. Filtration, followed by concentration, and silica gel chromatography affords the desired 4-(3-Acetyl-indol-1-yl)-2-bromo-benzonitrile as a tan solid (0.92 g, 36%)

The benzonitrile (0.15 mmol, 52 mg) is hydrolyzed by the method of example 1, using DMSO (4 drops), abs. ethanol (1 mL), KOH (157 mg), and 30% hydrogen peroxide (ca. 0.5 mL). Upon workup and chromatography the desired 4-(3-Acetyl-indol-1-yl)-2-bromo-benzamide is obtained as a yellow solid (5 mg, 10%). LCMS M+H=357.

### Example 9

### 3-Chloro-4-(3-isobutyryl-indol-1-yl)-benzamide (Compound 9)

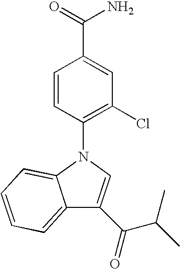

Sodium Hydride (60% oil suspension, 28 mmol, 1.12 g) is triturated with hexane and suspended in N,N-dimethylformamide (18 mL). 1-(1H-Indol-3-yl)-2-methyl-propan-1-one (20 mmol, 2.34 g) is added to the ice-cooled suspension. After 5 minutes, 3-chloro, 4-fluorobenzonitrile (28 mmol, 4.37 g) is added. The reaction is stirred at 45 degrees Celsius for 90 minutes. The reaction mixture is allowed to cool and is extracted into ethyl acetate (400 mL) and is washed with water (200 mL). The organic phase is dried over magnesium sulfate. Filtration, followed by concentration, and silica gel chromatography to afford the desired 3-Chloro-4-indol-1-yl-benzonitrile as a white solid (5.78 g, ˜quant) contaminated with some 3-chloro,4-fluorobenzonitrile. The product is used without further purification in the next step.

The crude benzonitrile (2 mmol, 504 mg) is dissolved in nitromethane (2 mL). Isobutyric anhydride (3 mmol, 0.49 mL) is added, followed by ytterbium triflate (0.5 mmol, 310 mg). The mixture is stirred for 1.5 h at 50 degrees Celsius, then cooled and extracted with methylene chloride (200 mL)/water (100 mL). The organic layer is collected, dried over magnesium sulfate, filtered, and concentrated. Chromatography to afford 530 mg (32%) of 3-Chloro-4-(3-isobutyryl-indol-1-yl)-benzonitrile as a thick gum.

3-Chloro-4-(3-isobutyryl-indol-1-yl)-benzonitrile (1.6 mmol, 527 mg) is hydrolyzed by the method of example 1, using DMSO (0.1 mL), abs. ethanol (4 mL), KOH (500 mg), and 30% hydrogen peroxide (ca. 2 mL). Upon workup and chromatography the desired 3-Chloro-4-(3-isobutyryl-indol-1-yl)-benzamide is obtained as a white foam (355 mg, 65%). LCMS M+H=341.

### Example 10

### 9-[4-(2-methoxy-ethylamino)-2-trifluoromethyl-phenyl]-1,2,3,9-tetrahydro-carbazol-4-one (Compound 10)

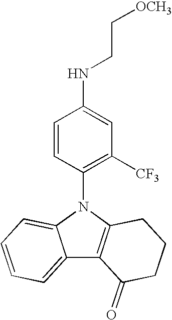

Sodium Hydride (60% oil suspension, 5 mmol, 200 mg) is triturated with hexane and suspended in N,N-dimethylformamide (4 mL). 1,2,3,9-Tetrahydro-carbazol-4-one (2.5 mmol, 462 mg) is added to the water-cooled suspension. After 5 minutes, 5-bromo-2-trifluorobenzotrifluoride (5 mmol, 0.71 g) is added. The reaction is stirred at 85 degrees Celsius for 3.5 h. The reaction mixture is allowed to cool and is extracted into ethyl acetate (200 mL) and is washed with water (50 mL). The organic phase is dried over magnesium sulfate. Filtration, followed by concentration, and silica gel chromatography affords the desired 9-(4-Bromo-2-trifluoromethyl-phenyl)-1,2,3,9-tetrahydro-carbazol-4-one as a brown foam (0.74 g, 73%).

9-(4-Bromo-2-trifluoromethyl-phenyl)-1,2,3,9-tetrahydro-carbazol-4-one (0.25 mmol, 102 mg), palladium acetate (15 mg), 1,1′-bis(diphenylphosphino)ferrocene (DPPF) (28 mg), methoxyethylamine (2 mmol, 0.17 mL), sodium t-butoxide (1 mmol, 96 mg), and toluene are combined in a sealed tube and microwaved at 110 degrees Celsius for 900 seconds at high absorbance. The reaction mixture is taken up in ethyl acetate (200 mL) and washed with water (50 mL). The organic layer is dried over magnesium sulfate, filtered, concentrated, and subjected to chromatography, affording the desired 9-[4-(2-methoxy-ethylamino)-2-trifluoromethyl-phenyl]-1,2,3,9-tetrahydro-carbazol-4-one as a solid (32 mg, 32%). LCMS M+H=403.

### Example 11

### 9-(4-Amino-2-chloro-phenyl)-1,2,3,9-tetrahydro-carbazol-4-one (Compound 11)

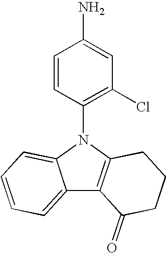

1,2,3,9-Tetrahydro-carbazol-4-one (2 g) is dissolved in anhydrous DMF (50 ml). Then, 4-fluoro-3-chloro-nitrobenzene (4.2 g, 2 eq.) is added. After the addition of Cs₂CO₃ (7 g, 2 eq.), the reaction mixture is stirred at 50° C. over night. The reaction mixture is diluted with ethyl acetate (200 ml), washed with brine and then water, dried over MgSO₄. After the solvent is rotavaped off, the residue purified by chromatography with ethyl acetate and hexane as eluent. The desired 9-(2-Chloro-4-nitro-phenyl)-1,2,3,9-tetrahydro-carbazol-4-one (3.49 g) is obtained.

9-(2-Chloro-4-nitro-phenyl)-1,2,3,9-tetrahydro-carbazol-4-one (3 g) is dissolved in ethyl acetate (200 ml), then 10% Pd/C (0.6 g) is added, and the flask is fitted with a hydrogen balloon and stirred at room temperature for 3 days. Then the reaction mixture is filtered through celite and washed with ethyl acetate. The filtrate is concentrated down and dried under vacuum. The crude 9-(4-Amino-2-chloro-phenyl)-1,2,3,9-tetrahydro-carbazol-4-one is obtained (2.9 g) and is used for next steps without further purification. LCMS M+H=311.

### Example 12

### N-[3-Chloro-4-(4-oxo-1,2,3,4-tetrahydro-carbazol-9-yl)-phenyl]-acetamide (Compound 12)

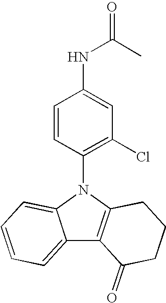

To 9-(4-Amino-2-chloro-phenyl)-1,2,3,9-tetrahydro-carbazol-4-one (0.1 mmol, 34 mg) is added, sequentially, acetonitrile (1 mL), pyridine (0.3 mmol, 0.02 mL), and acetic anhydride (0.3 mmol, 0.03 mL). The mixture is stirred at ambient temperature for 2 h, then taked up in ethyl acetate (50 mL) and washed with water (50 mL). The organic layer is dried over magnesium sulfate, filtered, concentrated, and chromatographed, to afford the desired N-[3-Chloro-4-(4-oxo-1,2,3,4-tetrahydro-carbazol-9-yl)-phenyl]-acetamide (25 mg, 71%) as a white solid. LCMS M+H=353.

### Example 13

### 1-(3-chloro-4-(4-oxo-3,4-dihydro-1H-carbazol-9(2H)-yl)phenyl)urea (Compound 13)

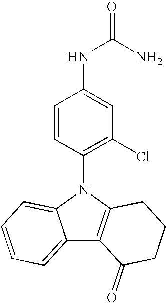

9-(4-Amino-2-chloro-phenyl)-1,2,3,9-tetrahydro-carbazol-4-one (1.2 g) is dissolved in acetic acid (100 ml), then KNCO (4 g) and water (20 ml) is added. The reaction mixture is stirred overnight for 2 days, and then rotavaped to dryness. The residue is diluted with ethyl acetate, washed with aq. NaHCO₃ solution and water, and dried over MgSO₄. After filtration and evaporation of solvent, the residue is purified by flash chromatography. 1-(3-chloro-4-(4-oxo-3,4-dihydro-1H-carbazol-9(2H)-yl)phenyl)urea (0.85 g) is obtained and its structure confirmed by analytical data. LCMS M+H=354.

### Example 14

### 3-(4-Oxo-1,2,3,4-tetrahydro-carbazol-9-yl)-4-trifluoromethyl-benzamide (Compound 14)

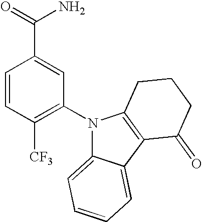

Sodium Hydride (60% oil suspension, 5 mmol, 200 mg) is triturated with hexane and suspended in N,N-dimethylformamide (4 mL). 1,2,3,9-Tetrahydro-carbazol-4-one (2.5 mmol, 462 mg) is added to the water-cooled suspension. After 5 minutes, 3-fluoro, 4-(trifluoromethyl)benzonitrile (3.5 mmol, 662 mg) is added. The reaction is maintained at 70 degrees Celsius for 30 minutes. Upon cooling, the mixture is extracted into ethyl acetate (200 mL) and is washed with water (50 mL). The organic phase is dried over magnesium sulfate. Filtration, followed by concentration, and silica gel chromatography affords 3-(4-Oxo-1,2,3,4-tetrahydro-carbazol-9-yl)-4-trifluoromethyl-benzonitrile as a brown solid (267 mg, 30%)

To 3-(4-Oxo-1,2,3,4-tetrahydro-carbazol-9-yl)-4-trifluoromethyl-benzonitrile (0.69 mmol, 246 mg) is added DMSO (0.1 mL), abs. ethanol (24 mL), and KOH (271 mg). The mixture is loared into a 50 degree Celsius oil bath, and 30% hydrogen peroxide (0.5 mL) is added. After 30 minutes, the reaction is taken up in ethyl acetate (150 mL) and washed with water (50 mL). The organic phase is dried using magnesium sulfate and filtered through a short plug of silica. Concentration affords 112 mg of the desired 3-(4-Oxo-1,2,3,4-tetrahydro-carbazol-9-yl)-4-trifluoromethyl-benzamide as a tan solid (44%). LCMS M+H=373.

### Example 15

### 3-Chloro-4-[3-(1-hydroxy-ethyl)-indol-1-yl]-benzamide (Compound 15)

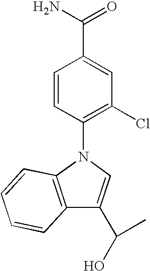

To methanol (3 mL) is added (0.72 mmol, 27 mg) of NaBH₄. The solution is stirred for five minutes before a solution of 4-(3-Acetyl-indol-1-yl)-3-chloro-benzamide (0.60 mmol, 188 mg) in methanol (3 mL) is added. The reaction is allowed to stir for 16 h, then additional NaBH₄ (0.60 mmol, 188 mg) is added. The reaction is stirred for an additional 3 h at room temp. The reaction is first diluted with water (5 mL) and then quenched with a few drops of 2N HCl until the solution reached a pH of approximately 6. The product is extracted with EtOAc (3×30 mL). The layers are separated and the organic layers are combined and washed with brine (1×50 mL) and then dried over magnesium sulfate. The solution is filtered by gravity and the solvent removed under reduced pressure. The residue is adsorbed onto silica gel and purified through column chromatography using 5% MeOH in methylene chloride, yielding 137 mg (72%) of the desired 3-Chloro-4-[3-(1-hydroxy-ethyl)-indol-1-yl]-benzamide as a white powder. LCMS M+H=315.

### Example 16

### 1-[1-(4-Amino-2-chloro-phenyl)-5-chloro-1H-indol-3-yl]-ethanone (Compound 16)

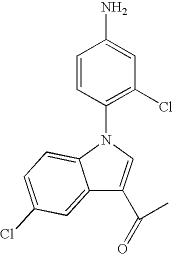

In a 20 mL reinforced microwave vial, 5-chloroindole (7.0 mmol, 1.06 g), 3-chloro-4-fluoronitrobenzene (7.7 mmol, 1.35 g) and potassium carbonate (14 mmol, 1.93) are combined dry. To this is added reagent-grade dimethylformamide (12 mL), resulting in a yellow solution. The vial is sealed and briefly agitated at ambient temperature. The reaction is heated in the microwave at 150 degrees Celsius for 900 sec, fixed hold time on, absorbance set to very high. The reaction is air-cooled and then poured into water (10 mL). The product is extracted with EtOAc (4×50 mL). The layers are separated and the organic layers combined, washed with brine (2×100 mL) and dried over sodium carbonate. The solution is filtered by gravity and the solvent removed under reduced pressure. The residue is adsorbed onto silica gel and purified through column chromatography using 10% EtOAc in hexanes as eluent. 1.41 g of 5-Chloro-1-(2-chloro-4-nitro-phenyl)-1H-indole is isolated as a shiny yellow powder (60%).

In a 100 mL round-bottom flask, 5-Chloro-1-(2-chloro-4-nitro-phenyl)-1H-indole (3.9 mmol, 1.22 g) is dissolved in nitromethane (5 mL). To this is added acetic anhydride (5.9 mmol, 0.57 mL). The solution is then heated to 50 degrees Celsius and is stirred for approximately 5 min. To this heated solution is added ytterbium(III)triflate (2 mmol, 1.24 g). The reaction is stirred at 50 degrees Celsius for 1.5 h and is then cooled to room temperature. The solution is diluted with methylene chloride (5 mL) and then poured into saturated ammonium chloride solution. The product is extracted with methylene chloride (3×50 mL). The layers are separated and the organic layers combined and dried over magnesium sulfate. The solution is filtered by gravity and the drying agent is washed with EtOAc. Solvent is removed under reduced pressure to afford spectroscopically pure 1-[5-Chloro-1-(2-chloro-4-nitro-phenyl)-1H-indol-3-yl]ethanone, which is isolated as a yellow-brown solid (1.33 g, 97%).

Reagent-grade methanol (10 mL) is added to of 10% palladium on carbon (155 mg). To this is added 1-[5-Chloro-1-(2-chloro-4-nitro-phenyl)-1H-indol-3-yl]ethanone (1.44 mmol, 508 mg) dissolved in methanol (10 mL). A hydrogen atmosphere is added and the reaction allowed to stir overnight at ambient temperature, recharging the hydrogen atmosphere as needed. The reaction is then filtered through a pad of Celite filter agent and washed with methanol (ca. 125 mL). The solvent is then removed under reduced pressure, resulting in a colorless oil. The oil is washed with diethyl ether and dried on the high vacuum, yielding an off-white shiny powder of 1-[1-(4-Amino-2-chloro-phenyl)-5-chloro-1H-indol-3-yl]-ethanone (452 mg, 98% yield). LCMS M+H=319.

### Example 17

### [4-(3-Acetyl-5-chloro-indol-1-yl)-3-chloro-phenyl]-urea (Compound 17)

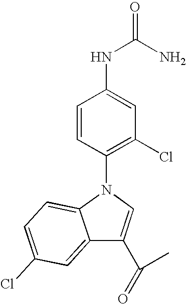

In a 25 mL round-bottom flask, 1-[1-(4-Amino-2-chloro-phenyl)-5-chloro-1H-indol-3-yl]-ethanone (0.39 mmol, 104 mg) is combined dry with sodium cyanate (3.3 mmol, 212 mg). To this is added acetic acid (4 mL) and H₂O (1 mL). The reaction is allowed to stir for two days at ambient temperature. The solution is diluted with water (5 mL) and extracted with methylene chloride (3×25 mL). The layers are separated and the organic layers combined, then washed with brine (1×50 mL). After drying with magnesium sulfate, the solution is filtered and the solvent removed under reduced pressure. The residue is then dissolved in EtOAc (0.5 mL) and recrystallized with hexanes. 10 mg (9% yield) of the titled [4-(3-Acetyl-5-chloro-indol-1-yl)-3-chloro-phenyl]-urea as an off-white solid is isolated. LCMS M+H=362.

### Example 18

### 1-[1-(4-Amino-2-chloro-phenyl)-6-chloro-1H-indol-3-yl]-ethanone (Compound 18)

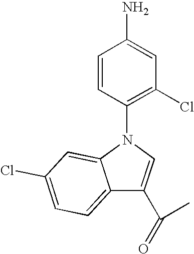

In a 20 mL reinforced microwave vial; 6-chloroindole (6.1 mmol, 0.92 g), 3-chloro-4-fluoronitrobenzene (0.67 mmol, 1.17 g), and potassium carbonate (12 mmol, 1.68 g) are combined dry. To this is added reagent-grade dimethylformamide (12 mL). The vial is sealed and agitated at ambient temperature. The reaction is heated in the microwave at 150 degrees Celsius for 900 sec, fixed hold time on, absorbance set to very high. The reaction is air-cooled and then poured into water (10 mL). The product is extracted with EtOAc (4×50 mL). The layers are separated and the organic layers combined, washed with brine (2×100 mL) and dried over sodium carbonate. The solution is filtered by gravity and the solvent removed under reduced pressure. The residue is adsorbed onto silica gel and purified through column chromatography using 10% EtOAc in hexanes as eluent. The desired 6-Chloro-1-(2-chloro-4-nitro-phenyl)-1H-indole is isolated as shiny yellow powder (1.34 g, 64%).

6-Chloro-1-(2-chloro-4-nitro-phenyl)-1H-indole (5.5 mmol, 1.17 g) is dissolved in nitromethane (5 mL). To this is added acetic anhydride (5.6 mmol, 0.54 mL). The solution is then heated to 50 degrees Celsius and is stirred for approximately 5 min. To this heated solution is added ytterbium(III)triflate (1.9 mmol, 1.18 g). The reaction is stirred at 50 degrees Celsius for 1.5 h and is then cooled to room temperature. The solution is diluted with methylene chloride (5 mL) and then poured into saturated ammonium chloride solution. The product is extracted with methylene chloride (3×50 mL). The layers are separated and the organic layers combined and dried over magnesium sulfate. The solution is filtered by gravity and the drying agent is washed with EtOAc. Solvent is removed under reduced pressure to afford the desired 1-[6-Chloro-1-(2-chloro-4-nitro-phenyl)-1H-indol-3-yl]ethanone as a yellow-brown solid (1.28 g, 96%).

In a 100 mL round-bottom flask under N₂, reagent-grade methanol (10 mL) is added to 159 mg of 10% palladium on carbon. To this is added the 1-[6-Chloro-1-(2-chloro-4-nitro-phenyl)-1H-indol-3-yl]ethanone (1.5 mmol, 522 mg) dissolved in reagent-grade methanol (10 mL). A hydrogen atmosphere is introduced and the reaction allowed to stir for two days at ambient temperatures, recharging the hydrogen as needed. The reaction is then filtered through a pad of Celite filter agent and washed with methanol (ca. 125 mL). The solvent is then removed under reduced pressure, resulting in a colorless oil. The oil is washed with diethyl ether and dried on the high vacuum, yielding 469 mg of the desired 1-[1-(4-Amino-2-chloro-phenyl)-6-chloro-1H-indol-3-yl]-ethanone as an off-white shiny powder (97%). LCMS M+H=319.

### Example 19

### [4-(3-Acetyl-6-chloro-indol-1-yl)-3-chloro-phenyl]-urea (Compound 19)

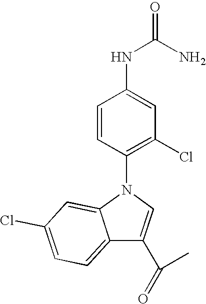

In a 25 mL round-bottom flask, 1-[1-(4-Amino-2-chloro-phenyl)-6-chloro-1H-indol-3-yl]-ethanone (0.32 mmol, 101 mg) is combined dry with sodium cyanate (3.2 mmol, 207 mg). To this is added acetic acid (4 mL) and H₂O (1 mL). The reaction is allowed to stir for two days at ambient temperature. The solution is diluted with H₂O (5 mL) and extracted with methylene chloride (3×25 mL). The layers are separated and the organic layers combined, washed with brine (1×50 mL) and the solvent removed under reduced pressure. The residue is then dissolved in EtOAc (0.5 mL) and recrystallized with hexanes. The expected [4-(3-Acetyl-6-chloro-indol-1-yl)-3-chloro-phenyl]-urea is isolated as an off-white solid (50 mg, 43%). LCMS M+H=362.

### Example 20

The following compounds are prepared essentially according to the procedures set forth in the preceding Schemes and examples.

| Compound | Structure | Name |
|---|---|---|
| 20 | 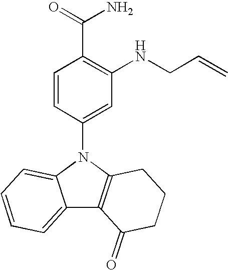 | 2-Allylamino-4-(4-oxo-1,2,3,4- tetrahydro-carbazol-9-yl)- benzamide M + H = 360 |
| 21 | 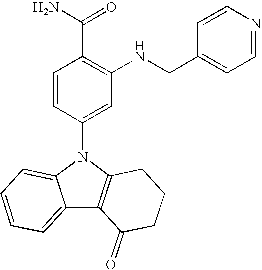 | 4-(4-Oxo-1,2,3,4-tetrahydro- carbazol-9-yl)-2-[(pyridin-4- ylmethyl) -amino]-benzamide M + H = 411 |
| 22 | 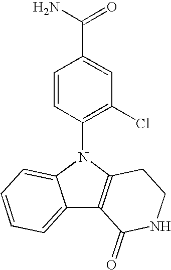 | 3-Chloro-4-(1-oxo-1,2,3,4- tetrahydro-pyrido[4,3-b]indol-5- yl)-benzamide M + H = 340 |
| 23 | 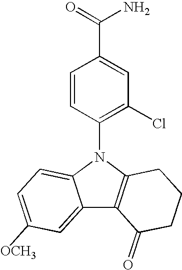 | 3-Chloro-4-(6-methoxy-4-oxo- 1,2,3,4-tetrahydro-carbazol-9- yl)-benzamide M + H = 369 |
| 24 | 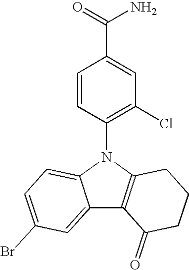 | 4-(6-Bromo-4-oxo-1,2,3,4- tetrahydro-carbazol-9-yl)-3- chloro-benzamide M + H = 417 |
| 25 | 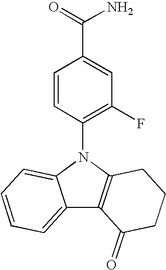 | 3-Fluoro-4-(4-oxo-1,2,3,4- tetrahydro-carbazol-9-yl)- benzamide M + H = 323 |
| 26 | 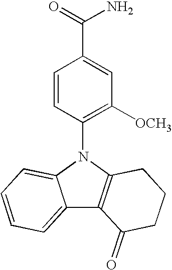 | 3-Methoxy-4-(4-oxo-1,2,3,4- tetrahydro-carbazol-9-yl)- benzamide M + H = 335 |
| 27 | 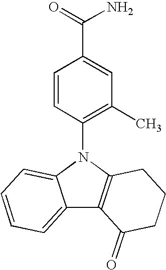 | 3-Methyl-4-(4-oxo-1,2,3,4- tetrahydro-carbazol-9-yl)- benzamide M + H = 319 |
| 28 | 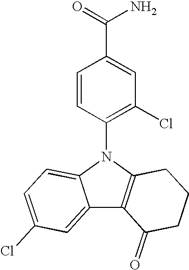 | 3-Chloro-4-(6-chloro-4-oxo- 1,2,3,4-tetrahydro-carbazol-9- yl)-benzamide M + H = 373 |
| 29 | 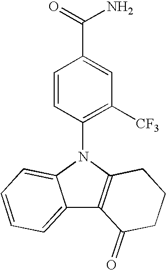 | 4-(4-Oxo-1,2,3,4-tetrahydro- carbazol-9-yl)-3- trifluoromethyl-benzamide M + H = 373 |
| 30 | 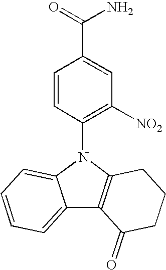 | 3-Nitro-4-(4-oxo-1,2,3,4- tetrahydro-carbazol-9-yl)- benzamide M + H = 350 |
| 31 | 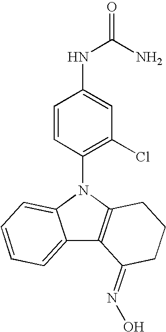 | [3-Chloro-4-(4-hydroxyimino- 1,2,3,4-tetrahydro-carbazol-9- yl)-phenyl]-urea M + H = 369 |
| 32 | 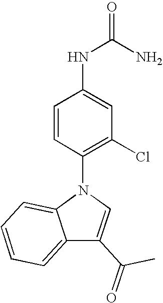 | [4-(3-Acetyl-indol-1-yl)-3- chloro-phenyl]-urea M + H = 328 |
| 33 | 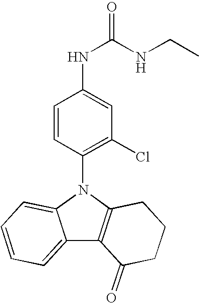 | 1-[3-Chloro-4-(4-oxo-1,2,3,4- tetrahydro-carbazol-9-yl)- phenyl]-3-ethyl-urea M + H = 382 |
| 34 | 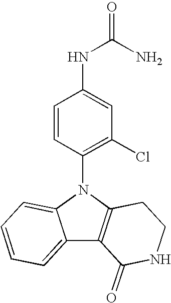 | [3-Chloro-4-(1-oxo-1,2,3,4- tetrahydro-pyrido[4,3-b]indol-5- yl)-phenyl]-urea M + H = 355 |
| 35 | 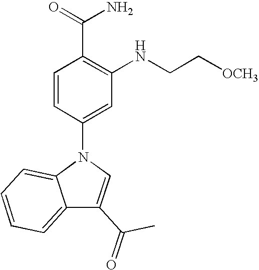 | 4-(3-Acetyl-indol-1-yl)-2-(2- methoxy-ethylamino)-benzamide M + H = 352 |
| 36 | 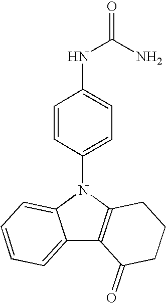 | [4-(4-Oxo-1,2,3,4-tetrahydro- carbazol-9-yl)-phenyl]-urea M + H = 320 |
| 37 | 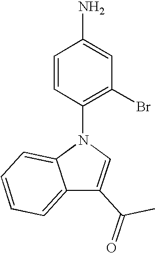 | 1-[1-(4-Amino-2-bromo-phenyl)- 1H-indol-3-yl]-ethanone M + H = 329 |
| 38 | 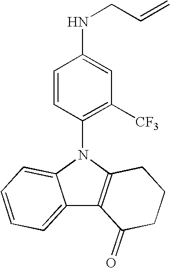 | 9-(4-Allylamino-2- trifluoromethyl-phenyl)-1,2,3,9- tetrahydro-carbazol-4-one M + H = 385 |
| 39 | 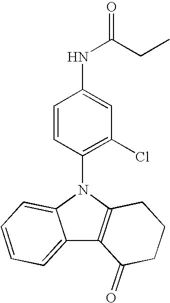 | N-[3-Chloro-4-(4-oxo-1,2,3,4- tetrahydro-carbazol-9-yl)- phenyl]-propionamide M + H = 367 |
| 40 | 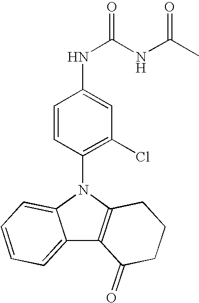 | 1-Acetyl-3-[3-chloro-4-(4-oxo- 1,2,3,4-tetrahydro-carbazol-9- yl)-phenyl]-urea M + H = 396 |
| 41 | 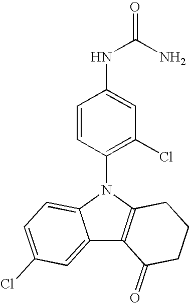 | [3-Chloro-4-(6-chloro-4-oxo- 1,2,3,4-tetrahydro-carbazol-9- yl)-phenyl]-urea M + H = 388 |
| 42 | 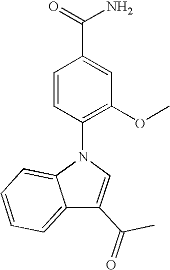 | 4-(3-Acetyl-indol-1-yl)-3- methoxy-benzamide M + H = 309 |
| 43 | 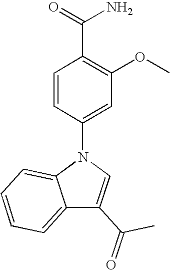 | 4-(3-Acetyl-indol-1-yl)-2- methoxy-benzamide M + H = 309 |
| 44 | 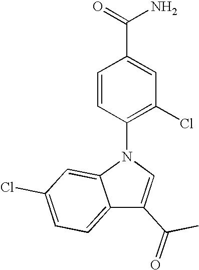 | 4-(3-Acetyl-6-chloro-indol-1- yl)-3-chloro-benzamide M + H = 347 |
| 45 | 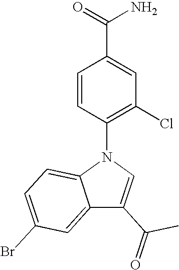 | 4-(3-Acetyl-5-bromo-indol-1-yl)- 3-chloro-benzamide M + H = 390 |
| 46 | 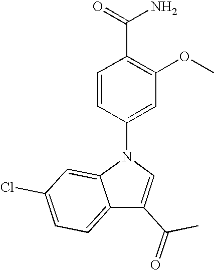 | 4-(3-Acetyl-6-chloro-indol-1- yl)-2-methoxy-benzamide M + H = 343 |
| 47 | 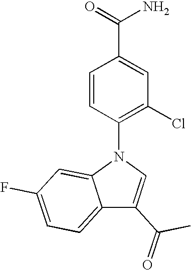 | 4-(3-Acetyl-6-fluoro-indol-1- yl)-3-chloro-benzamide M + H = 331 |

### Biological Evaluation

Cell Proliferation Assays

A panel of cancer cell lines is obtained from the DCTP Tumor Repository, National Cancer Institute (Frederick, Md.) or ATCC (Rockville, Md.). Cell cultures are maintained in Hyclone RPMI 1640 medium (Logan, Utah) supplemented with 10% fetal bovine serum and 20 mM HEPES buffer, final pH 7.2, at 37° C. with a 5% CO₂ atmosphere. Cultures are maintained at sub-confluent densities.

For proliferation assays, cells are seeded with the appropriate medium into 96 well plates at 1,000-2,500 cells per well, depending on the cell line, and are incubated overnight. The following day, test compound, DMSO solution (negative control), or Actinomycin D (positive control) is added to the appropriate wells as 10× concentrated stocks prepared in phosphate buffered saline. The cell plates where then incubated for an additional 2-5 days, depending on the cell line, to allow proliferation to occur. To measure cell density, growth medium is removed from the adherent cell lines, and the plates stored at −80° C. Using these assay plates, relative amounts of DNA in each well is determined using the Cyquant DNA assay kit from R&D Systems (Eugene, Oreg.) following the manufacturer's directions. Results for each compound treatment are compared to DMSO vehicle control (100%) and 10 μM Actinomycin D treated cells (0%).

Several representative compounds of the invention are listed below and the range within which their respective inhibitory activities against PC-3 cell proliferation falls is shown, where +++ stands for an IC₅₀ value that is less than 0.5 μM, ++ between 0.5 and 5 μM, + between 5 and 50 μM.

| | |
|---|---|
| 2 | +++ |
| 4 | ++ |
| 5 | +++ |
| 6 | +++ |
| 7 | +++ |
| 10 | ++ |
| 13 | +++ |
| 14 | + |
| 20 | ++ |
| 22 | ++ |

Analysis of Cell Cycle Status

Cells are seeded onto 96-well Packard View plates and incubated overnight. Test compounds are added to individual wells the following day at 10× concentrations and the plates returned to the incubator.

For identification of compounds that blocked cell cycle progression in M-phase, cells are incubated with compounds for 5 hours. After incubation, the cell growth media is removed and the cell monolayers are fixed with 3.7% formaldehyde, followed by treatment with 0.1% Triton X-100, and probed with a monoclonal phospho-specific Histone 3 serine 10 antibody. Bound primary antibody is detected with a FITC or TRITC conjugated secondary antibody. The cellular DNA is stained using Hoechst dye. Fixed cells are visualized using an ArrayScan 4.5 HCS plate reader. Cells positive for antibody binding are identified and quantified using the Target Activation Algorithm. IC₅₀ Data from test compounds is generated by comparing test compound activity to the percentage of cells in M-phase detected in 500 nM vinblastine treated cells (100% M-phase block), and the percentage of M-phase cells detected in the DMSO control treated cells (0% M-phase block).

For determining the percentage of cells in the G1 or G2 phase of the cell cycle, cells are treated for 24 hours. After treatment, the cell monolayers are fixed with 3.7% formaldehyde, followed by treatment with 0.1% Triton X-100, and probed with a monoclonal phospho-specific Histone 3 serine 10 antibody. Bound primary antibody is detected with a FITC or TRITC conjugated secondary antibody. The cellular DNA is stained using Hoechst dye. Fixed cells are visualized using an ArrayScan 4.5 HCS plate reader. The Cell Cycle Analysis algorithm is used to quantify the DNA content and phospho-Histone3 serine 10 status of individual cells. The data is used to generate histograms for assignment to G1, S and G2 cell cycle phase.

Several representative compounds of the invention are listed below and the range within which their respective M-phase block activities is shown, where +++ stands for an IC₅₀ value that is less than 0.5 μM, ++ between 0.5 and 5 μM, + between 5 and 50 μM.

| | |
|---|---|
| 17 | ++ |
| 18 | ++ |
| 29 | +++ |
| 35 | ++ |
| 37 | + |
| 38 | ++ |
| 40 | +++ |
| 41 | +++ |
| 43 | +++ |
| 44 | +++ |

Tubulin Polymerization

Cells are seeded onto 96-well Packard View plates and incubated overnight. Test compounds are added to individual wells the following day at 10× concentrations and the plates returned to the incubator.

For identifying compounds which disrupt the cellular cytoskeleton network, cell treated for various time points are fixed with 3.7% formaldehyde, followed by treatment with 0.1% Triton X-100, and probed with a Cy3-conjugated β-tubulin specific antibody. The cellular DNA is stained using Hoechst dye. Cells are imaged using an ArrayScan 4.5 HCS plate reader. Images from compound treated cells are compared to DMSO treated cells and the extent of cytoskeleton staining determined.

Compound interference with tubulin polymerization rates is measured using the Tubulin Polymerization Assay Kit (cat.#BK011) from Cytoskeleton, Inc., following the standard protocol.

Preferred compounds of the invention have IC₅₀ values of less than 20 uM in the tubulin polymerization assays described above.

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

What is claimed is:

1. A compound of the formula: 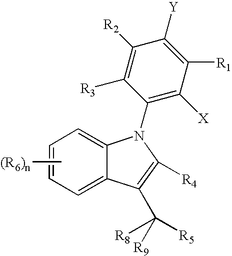
or a pharmaceutically acceptable salt thereof, wherein
X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆)alkylamino, mono C₃-C₆ alkenylamino, carboxamide, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
where each of the foregoing aryl and heteroaryl groups is optionally substituted with from 1-4 R₂₂ groups;
where R₂₂ at each occurrence is independently C₁-C₆ alkyl, C₁-C₆ alkoxy, trifluoromethyl, halogen, hydroxy, amino, mono- or di-(C₁-C₆)alkylamino, nitro, halo(C₁-C₆)alkyl, halo(C₁-C₆)alkoxy, or carboxamide;
or R₂ and R₃ together with the atoms to which they are attached form a 5-12 membered mono-, bi-, or tricyclic ring system, where the 5-12 membered ring is partially unsaturated or aromatic and optionally contains one or two of oxygen, S(O)ₘ where m is 0, 1, or 2, nitrogen, or NR₇ where R₇ is hydrogen or C₁-C₆ alkyl, and wherein the 5-12 membered ring is optionally substituted with 1 or 2 R₂₂ groups;
or R₁ and X together with the atoms to which they are attached form a 5-12 membered mono-, bi-, or tricyclic ring system, where the 5-12 membered ring is partially unsaturated or aromatic and optionally contains one or two of oxygen, S(O)ₘ where m is 0, 1, or 2, nitrogen, or NR₇ where R₇ is hydrogen or C₁-C₆ alkyl, and wherein the 5-12 membered ring is optionally substituted with 1 or 2 R₂₂ groups;
Y is H, NR₇R₇′, NR₇(C₃-C₆)alkenyl, NR₇CONHCOR₇′, C(=Z)NR₇R₇′, NR₇CONR₇R₇′, NR₇COR₇′, or —NR₇—(C₁-C₆)alkyl-(C₁-C₆)alkoxy, where Z is O, S, or NOR₇, and
R₇ and R₇′ at each occurrence are independently selected from H and C₁-C₆ alkyl;
R₄ is H or C₁-C₆ alkyl optionally substituted with 1-2 groups selected from oxo, aryl, heteroaryl, or R₂₂;
R₅ is OR₇, NR₇R₇′, NR₇OR₇′, or C₁-C₆ alkyl optionally substituted with 1-2 groups selected from oxo, aryl, heteroaryl, or R₂₂; or
R₄ and R₅ together with the atoms to which they are attached form a 5-12 membered mono-, bi-, or tricyclic ring system, where the 5-12 membered ring is partially unsaturated or aromatic and optionally contains one or two of oxygen, S(O)ₘ where m is 0, 1, or 2, nitrogen, NOR₇ or NR₇ where R₇ is hydrogen or C₁-C₆ alkyl, and wherein the 5-12 membered ring is optionally substituted with 1 or 2 R₂₂ groups;
n is 0, 1, 2, 3, or 4;
R₆ at each occurrence is independently halo, C₁-C₆ alkyl, halo (C₁-C₆)-alkyl, C₁-C₆ alkoxy, nitro, hydroxyl, cyano, alkenyl, alkynyl, thiol, amino, mono- or di-(C₁-C₆) alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
where each of the foregoing aryl groups and heteroaryl groups is optionally substituted with from 1-4 R₂₂ groups;
or two R₆ groups on adjacent carbons, together with the atoms to which they are attached, form a 5-12 membered mono-, bi-, or tricyclic ring system fused to the ring to which Y is attached, where the 5-12 membered ring is partially unsaturated or aromatic and optionally contains one or two of oxygen, S(O)ₘ where m is 0, 1, or 2, nitrogen, or NR₇ where R₇ is hydrogen or C₁-C₆ alkyl, wherein the 5-12 membered ring is optionally substituted with 1 or 2 R₂₂ groups;
R₈ is H; and
R₉ is OH;
or R₈ and R₉ together are Q, wherein Q is ═O or ═NOR₇, provided that when the 5-12 membered ring formed by R₄ and R₅ is aromatic, one of R₈ and R₉ is absent.

2. A compound according to claim 1 wherein X, R₁, R₂, and R₃ are each independently selected from H, halo, C₁-C₆ alkyl, halo (C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆) alkylamino, aryl, heteroaryl, C₃-C₇ cycloalkyl, and C₃-C₇ cycloalkyl(C₁-C₆)alkyl, where each alkyl or cycloalkyl group is optionally substituted with aryl or heteroaryl, where each of the foregoing aryl groups and heteroaryl groups is optionally substituted with from 1-4 R₂₂ groups.

3. A compound according to claim 1 wherein R₁, R₂, and R₃ are each hydrogen, halo, C₁-C₆ alkyl, C₁-C₆ alkoxy, or hydroxy. and X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino.

4. A compound according to claim 1 wherein R₄ is H or C₁-C₆ alkyl and R₅ is OR₇, NR₇R₇, NR₇OR₇, or C₁-C₆ alkyl.

5. A compound according to claim 1 wherein n is 0.

6. A compound according to claim 1 wherein n is 1 and R₆ is halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl or heteroaryl.

7. A compound according to claim 1 of the formula: 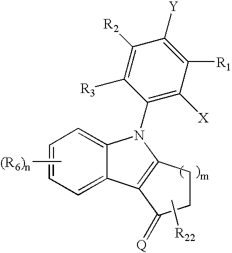
wherein
m is 1, 2, or 3; and
Q is O or NOR₇.

8. A compound according to claim 7 wherein X, R₁, R₂, and R₃ are each independently selected from H, halo, C₁-C₆ alkyl, halo (C₁-C₆)-alkyl, C₁-C₆ alkoxy, nitro, hydroxy, cyano, C₂-C₆ alkenyl, C₂-C₆ alkynyl, thiol, amino, mono- or di-(C₁-C₆) alkylamino, aryl, heteroaryl, C₃-C₇ cycloalkyl, and C₃-C₇ cycloalkyl(C₁-C₆)alkyl, where each alkyl or cycloalkyl group is optionally substituted with aryl or heteroaryl, where each of the foregoing aryl groups and heteroaryl groups is optionally substituted with from 1-4 R₂₂ groups.

9. A compound according to claim 7 wherein R₁, R₂, and R₃ are each hydrogen, halo, C₁-C₆ alkyl, C₁-C₆ alkoxy, or hydroxy. and X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino.

10. A compound according to claim 7 wherein m is 2.

11. A compound according to claim 7 wherein n is 0.

12. A compound according to claim 7 wherein n is 1 and R₆ is halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl or heteroaryl.

13. A compound according to claim 7 wherein Q is O.

14. A compound according to claim 7 wherein Q is NOH.

15. A compound according to claim 7 of the formula: 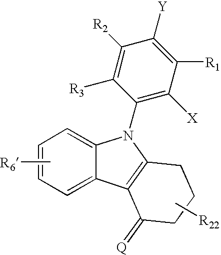
wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
where each of the foregoing aryl and heteroaryl groups is optionally substituted with from 1-4 R₂₂ groups.

16. A compound according to claim 7 of the formula: 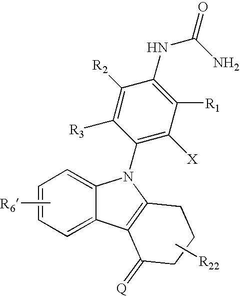
wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
where each of the foregoing aryl and heteroaryl groups is optionally substituted with from 1-4 R₂₂ groups.

17. A compound according to claim 7 of the formula: 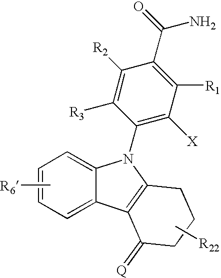
and pharmaceutically acceptable salts thereof, wherein
R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
where each of the foregoing aryl and heteroaryl groups is optionally substituted with from 1-4 R₂₂ groups.

18. A compound according to claim 7 of the formula: 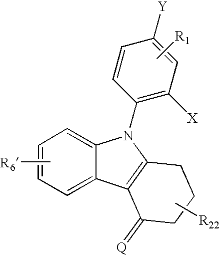
wherein R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
where each of the foregoing aryl and heteroaryl groups is optionally substituted with from 1-4 R₂₂ groups.

19. A compound according to claim 7 of the formula:

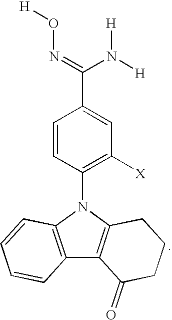

20. A compound according to claim 7 of the formula:

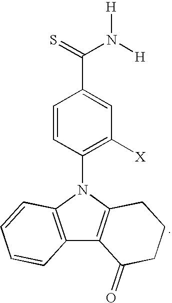

21. A compound according to claim 7 of the formula:

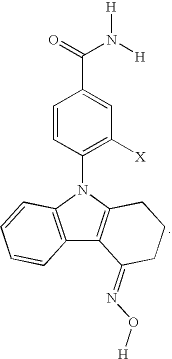

22. A compound according to claim 7 of the formula:

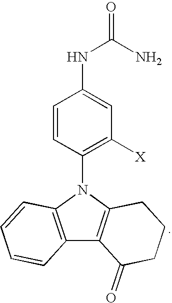

23. A compound according to claim 1, of the formula, 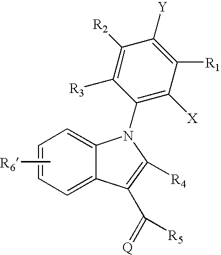
or pharmaceutically acceptable salts thereof, wherein
R₄ is H or C₁-C₆ alkyl;
R₅ is C₁-C₆ alkyl; and
R₆′ is H, halo, C₁-C₆ alkyl, halo(C₁-C₆)-alkyl, C₁-C₆ alkoxy, nitro, hydroxyl, cyano, alkenyl, alkynyl, thiol, amino, mono- or di-(C₁-C₆)alkylamino, aryl, heteroaryl, C₃-C₇ cycloalkyl, and C₃-C₇ cycloalkyl(C₁-C₆)alkyl, where each alkyl or cycloalkyl group is optionally substituted with aryl, heteroaryl, or 1-2 R₂₂ groups,
where each of the foregoing aryl and heteroaryl groups is optionally substituted with from 1-4 R₂₂ groups.

24. A compound according to claim 23, wherein
X, R₁, R₂, and R₃ are each independently selected from H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino.

25. A compound according to claim 23, wherein
R₁, R₂, and R₃ are each hydrogen, halo, or C₁-C₆ alkoxy and X is H, halo, nitro, C₁-C₆ alkyl, halo(C₁-C₆)-alkyl or C₁-C₆ alkoxy.

26. A compound according to claim 23, of the formula,

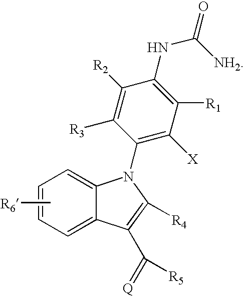

27. A compound according to claim 26, wherein
R₁, R₂, and R₃ are each hydrogen, halo, or C₁-C₆ alkoxy; and
X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino.

28. A compound according to claim 27, wherein
R₁, R₂, and R₃ are each hydrogen; and
X is H, halo or halo(C₁-C₆)-alkyl.

29. A compound according to claim 26, wherein
Q is O or N—OH.

30. A compound according to claim 23, of the formula,

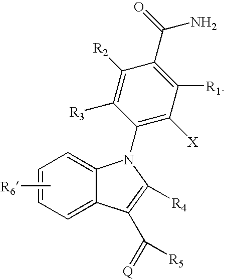

31. A compound according to claim 30, wherein
R₁, R₂, and R₃ are each hydrogen, halo, or C₁-C₆ alkoxy; and
X is H, halo, C₁-C₆ alkyl, halo(C₁-C₆)-alkyl, C₁-C₆ alkoxy, nitro, hydroxy, cyano, C₂-C₆ alkenyl, C₂-C₆ alkynyl, thiol, amino, or mono- or di-(C₁-C₆)alkylamino.

32. A compound according to claim 31, wherein
R₁, R₂, and R₃ are each hydrogen; and
X is H, halo or halo(C₁-C₆)-alkyl.

33. A compound according to claim 30, wherein
Q is O or N—OH.

34. A compound according to claim 1 which is
2-Allylamino-4-(4-oxo-1,2,3,4-tetrahydro-carbazol-9-yl)-benzamide;
3-Bromo-4-(4-oxo-1,2,3,4-tetrahydro-carbazol-9-yl)-benzamide;
9-[4-(Amino-hydroxyamino-methyl)-2-bromo-phenyl]-1,2,3,9-tetrahydro-carbazol-4-one;
3-Chloro-4-(4-oxo-1,2,3,4-tetrahydro-carbazol-9-yl)-benzamide;
4-(4-Oxo-1,2,3,4-tetrahydro-carbazol-9-yl)-2-[(pyridin-4-ylmethyl)-amino]-benzamide;
3-Chloro-4-(1-oxo-1,2,3,4-tetrahydro-pyrido[4,3-b]indol-5-yl)-benzamide;
3-Chloro-4-(6-methoxy-4-oxo-1,2,3,4-tetrahydro-carbazol-9-yl)-benzamide;
4-(6-Bromo-4-oxo-1,2,3,4-tetrahydro-carbazol-9-yl)-3-chloro-benzamide;
3-Fluoro-4-(4-oxo-1,2,3,4-tetrahydro-carbazol-9-yl)-benzamide;
3-Methoxy-4-(4-oxo-1,2,3,4-tetrahydro-carbazol-9-yl)-benzamide;
3-Methyl-4-(4-oxo-1,2,3,4-tetrahydro-carbazol-9-yl)-benzamide;
3-Bromo-4-(4-oxo-1,2,3,4-tetrahydro-carbazol-9-yl)-thiobenzamide;
3-Chloro-4-(6-chloro-4-oxo-1,2,3,4-tetrahydro-carbazol-9-yl)-benzamide;
3-Bromo-4-(4-hydroxyimino-1,2,3,4-tetrahydro-carbazol-9-yl)-benzamide;
4-(4-Oxo-1,2,3,4-tetrahydro-carbazol-9-yl)-3-trifluoromethyl-benzamide;
3-Nitro-4-(4-oxo-1,2,3,4-tetrahydro-carbazol-9-yl)-benzamide;
3-(4-Oxo-1,2,3,4-tetrahydro-carbazol-9-yl)-4-trifluoromethyl-benzamide;
9-(4-Amino-2-chloro-phenyl)-1,2,3,9-tetrahydro-carbazol-4-one;
4-(3-Acetyl-indol-1-yl)-3-chloro-benzamide;
[3-Chloro-4-(4-oxo-1,2,3,4-tetrahydro-carbazol-9-yl)-phenyl]-urea;
3-Chloro-4-[3-(1-hydroxyimino-ethyl)-indol-1-yl]-benzamide;
9-[4-(2-Methoxy-ethylamino)-2-trifluoromethyl-phenyl]-1,2,3,9-tetrahydro-carbazol-4-one;
[3-Chloro-4-(4-hydroxyimino-1,2,3,4-tetrahydro-carbazol-9-yl)-phenyl]-urea;
3-Chloro-4-(3-isobutyryl-indol-1-yl)-benzamide;
[4-(3-Acetyl-indol-1-yl)-3-chloro-phenyl]-urea;
1-[3-Chloro-4-(4-oxo-1,2,3,4-tetrahydro-carbazol-9-yl)-phenyl]-3-ethyl-urea;
[3-Chloro-4-(1-oxo-1,2,3,4-tetrahydro-pyrido[4,3-b]indol-5-yl)-phenyl]-urea;
3-Chloro-4-[3-(1-hydroxy-ethyl)-indol-1-yl]-benzamide;
4-(3-Acetyl-indol-1-yl)-2-bromo-benzamide;
4-(3-Acetyl-indol-1-yl)-2-(2-methoxy-ethylamino)-benzamide;
N-[3-Chloro-4-(4-oxo-1,2,3,4-tetrahydro-carbazol-9-yl)-phenyl]-acetamide;
[4-(4-Oxo-1,2,3,4-tetrahydro-carbazol-9-yl)-phenyl]-urea;
1-[1-(4-Amino-2-bromo-phenyl)-1H-indol-3-yl]-ethanone;
1-[1-(4-Amino-2-chloro-phenyl)-5-chloro-1H-indol-3-yl]-ethanone;
1-[1-(4-Amino-2-chloro-phenyl)-6-chloro-1H-indol-3-yl]-ethanone;
9-(4-Allylamino-2-trifluoromethyl-phenyl)-1,2,3,9-tetrahydro-carbazol-4-one;
[4-(3-Acetyl-5-chloro-indol-1-yl)-3-chloro-phenyl]-urea;
[4-(3-Acetyl-6-chloro-indol-1-yl)-3-chloro-phenyl]-urea;
N-[3-Chloro-4-(4-oxo-1,2,3,4-tetrahydro-carbazol-9-yl)-phenyl]-propionamide;
1-Acetyl-3-[3-chloro-4-(4-oxo-1,2,3,4-tetrahydro-carbazol-9-yl)-phenyl]-urea;
[3-Chloro-4-(6-chloro-4-oxo-1,2,3,4-tetrahydro-carbazol-9-yl)-phenyl]-urea;
4-(3-Acetyl-indol-1-yl)-3-methoxy-benzamide; 4-(3-Acetyl-indol-1-yl)-2-methoxy-benzamide;
4-(3-Acetyl-6-chloro-indol-1-yl)-3-chloro-benzamide;
4-(3-Acetyl-5-bromo-indol-1-yl)-3-chloro-benzamide;
4-(3-Acetyl-6-chloro-indol-1-yl)-2-methoxy-benzamide; or
4-(3-Acetyl-6-fluoro-indol-1-yl)-3-chloro-benzamide.

35. A pharmaceutical composition comprising at least one compound or salt according to claim 1 and a pharmaceutically acceptable solvent, carrier, excipient, adjuvant or a combination thereof.

36. A method of treating diseases and/or conditions related to cell proliferation and/or abnormal cell mitosis, such as cancer, inflammation and inflammation-associated disorders, and conditions associated with angiogenesis, the method comprising administering to a patient in need of such treatment a pharmaceutically acceptable amount of a compound or salt according to claim 1.

37. A method of treating diseases and/or conditions related to cell proliferation and/or abnormal cell mitosis, such as cancer, inflammation and inflammation-associated disorders, and conditions associated with angiogenesis, the method comprising administering to a patient in need of such treatment a pharmaceutically acceptable amount of a compound or salt according to claim 1.

38. A package comprising a compound of claim 1 in a container with instructions on how to use the compound.
